(19) United States
(12) Patent Application Publication
(10) Pub. No.: US 2025/0313633 A1
(43) Pub. Date: Oct. 9, 2025

(54) Anti-B7H3 antibody and Uses thereof

(71) Applicant: Wuxi Biologics Ireland Limited, Co Louth (IE)

(72) Inventors: Naiyan Rong, Shanghai (CN); Zhuozhi Wang, Shanghai (CN); Jie Li, Shanghai (CN); Jijie Gu, Shanghai (CN)

(73) Assignee: Wuxi Biologics Ireland Limited, Co Louth (IE)

(21) Appl. No.: 18/866,451

(22) PCT Filed: May 17, 2023
(86) PCT No.: PCT/CN2023/094711

§ 371 Date: Nov. 15, 2024

(30) Foreign Application Priority Data
May 18, 2022 (WO) PCT/CN2022/093524

(51) Int. Cl. C07K 16/28

(52) U.S. Cl. CPC C07K 16/2827; C07K 2317/24; C07K 2317/33; C07K 2317/40; C07K 2317/77; C07K 2317/92

(57) ABSTRACT

Provided are an antibody or antigen-binding portion thereof that binds B7H3, methods of producing the antibody, and methods of treating B7H3-related disorders using the antibody or antigen-binding portion thereof.

### CROSS REFERENCE

This application claims the benefit of International application PCT/CN2022/093524, filed on May 18, 2022, which is incorporated herein by reference in its entirety.

### SEQUENCE LISTING

The present application is filed with a Sequence Listing in electronic form. The entire contents of the Sequence Listing are hereby incorporated by reference.

### FIELD

The present disclosure generally relates to anti-B7H3 antibodies or antigen-binding portion thereof, a method for preparing the same and uses thereof.

### BACKGROUND

B7H3, also known as CD276 and B7RP-2, is a B7 family member and shares 20%-27% amino acid identity with other B7 family members. Although B7H3 transcript was ubiquitously expressed, it was limited and maintained at low level expression on normal tissue and immune cells, and was overexpressed in multiple human malignancies, including melanoma, breast cancer, prostate cancer etc. It was reported B7-H3 is expressed either on the membrane, in the cytoplasm, or within the nucleus of cancer cells but also on the tumor-associated vasculature. B7H3 was not constitutively expressed on T-cells, NK cells and APCs, including DCs and macrophages, but its expression can be induced on APCs by GM-CSF, IFNγ etc.

B7-H3 is a promising anti-cancer target. The precise function of B7-H3 remains unclear, as its different immune functions have been demonstrated, which includes stimulating, inhibiting T-cell proliferation and inhibiting NK cell function. It was assumed there are different receptors on immune cells which may compete binding to B7H3 on tumors. Besides its immune checkpoint function, it was reported high expression B7H3 level correlates with poorer prognosis in cancers and also enhances cell proliferation, migration, invasion, angiogenesis, metastatic capacity and anti-cancer drug resistance.

Like other B7 family molecules, B7H3 is a type-1 transmembrane glycoprotein whose ectodomain contains a IgV-IgC domain pair. Unlike mouse B7H3 gene which has only a single copy of IgV-IgC domain pair, human B7H3 was found with two isoforms. One isoform contains a single copy (named as 2IgB7H3), the other one has two IgV-IgC domain pairs (named as 4IgB7H3) that results from gene duplication and differential splicing. 4IgB7H3 rather than 2IgB7H3 is the major isoform expressed on immunocytes as well as malignant cells, which indicates 4IgB7H3 may play a distinctive important role in tumor development and cancer immunity.

As B7H3 was overexpressed on many tumor cells, therapeutic molecules that target B7H3 are being developed to treat related indications. However, the development of novel anti-B7H3 antibodies still has improvement space and clinical needs.

### SUMMARY

These and other objectives are provided for by the present disclosure which, in a broad sense, is directed to compounds, methods, compositions and articles of manufacture that provide antibodies with improved efficacy. The benefits provided by the present disclosure are broadly applicable in the field of antibody therapeutics and diagnostics and may be used in conjunction with antibodies that react with a variety of targets.

In one aspect, the disclosure provides an antibody or antigen-binding portion thereof that specifically binds to B7H3 antigen. The B7H3 antigen may be human B7H3, mouse B7H3 and/or cynomolgous monkey B7H3 or fragments (e.g. extracellular domain) thereof. Preferably, the B7H3 antigen is human 4IgB7H3 protein or extracellular domain thereof.

In some embodiments, the antibody or antigen-binding portion thereof as disclosed herein comprises:
a heavy chain CDR1 (HCDR1) comprising the amino acid sequence of SEQ ID No: 1; a HCDR2 comprising the amino acid sequence of SEQ ID No: 2; and a HCDR3 comprising the amino acid sequence of SEQ ID No: 3; and
a light chain CDR1 (LCDR1) comprising the amino acid sequence of SEQ ID No: 4 or 7; a LCDR2 comprising the amino acid sequence of SEQ ID No: 5, and a LCDR3 comprising the amino acid sequence of SEQ ID No: 6.

In some embodiments, the antibody or antigen-binding portion thereof as disclosed herein comprises:
a heavy chain CDR1 (HCDR1) comprising the amino acid sequence of SEQ ID No: 1; a HCDR2 comprising the amino acid sequence of SEQ ID No: 2; and a HCDR3 comprising the amino acid sequence of SEQ ID No: 3; and
a light chain CDR1 (LCDR1) comprising the amino acid sequence of KSSQSLLXISSNQKNYLA wherein X₁ may be any amino acid other than N; a LCDR2 comprising the amino acid sequence of SEQ ID No: 5, and a LCDR3 comprising the amino acid sequence of SEQ ID No: 6.

Preferably, the X₁ as described above is Q and the LCDR1 comprises the amino acid sequence of SEQ ID No: 4.

In some embodiments, the antibody or antigen-binding portion thereof as disclosed herein comprises:
a heavy chain CDR1 (HCDR1) comprising the amino acid sequence of SEQ ID No: 1; a HCDR2 comprising the amino acid sequence of SEQ ID No: 2; and a HCDR3 comprising the amino acid sequence of SEQ ID No: 3; and
a light chain CDR1 (LCDR1) comprising the amino acid sequence of KSSQSLLNX2SNQKNYLA wherein X may be any amino acid other than S; a LCDR2 comprising the amino acid sequence of SEQ ID No: 5, and a LCDR3 comprising the amino acid sequence of SEQ ID No: 6.

Preferably, the X₂ as described above is P and the LCDR1 comprises the amino acid sequence of SEQ ID No: 18.

In some embodiments, the antibody or antigen-binding portion thereof comprises a heavy chain variable region (VH) and a light chain variable region (VL), wherein the VH comprises:
(A) an amino acid sequence as set forth in SEQ ID No: 8 or 10;
(B) an amino acid sequence which is at least 85%, at least 90%, or at least 95% identical to SEQ ID No: 8 or 10; or
(C) an amino acid sequence with addition, deletion and/or substitution of one or more (for example, 1, 2, 3, 4, 5, 6, 7, 8, 9 or 10) amino acids in the framework region compared with SEQ ID No: 8 or 10;
and or the VL comprises:
(A) an amino acid sequence as set forth in SEQ ID No: 9 or 11;
(B) an amino acid sequence which is at least 85%, at least 90%, or at least 95% identical to SEQ ID No: 9 or 11; or
(C) an amino acid sequence with addition, deletion and/or substitution of one or more (for example, 1, 2, 3, 4, 5, 6, 7, 8, 9 or 10) amino acids in the framework region compared with SEQ ID No: 9 or 11.

In some embodiments, the antibody or antigen-binding portion thereof comprises VH and/or VL comprising one or more amino acid substitutions in the framework region. In some embodiments, at least one of the amino acid substitutions is a back mutation wherein an amino acid from human germline sequences is substituted by a different amino acid at a corresponding position in a parental antibody.

In some embodiments, the antibody or antigen-binding portion thereof comprises at least one amino acid modification (e.g. amino acid substitution) to remove potential post-translational modification site(s) or glycosylation site(s). Such amino acid modifications may be in the CDR region or the framework region.

In some embodiments, the antibody or antigen-binding portion thereof is a full-length antibody, ScFv, Fab, F(ab′)2, or Fv fragment.

In some embodiments, the antibody further comprises an immunoglobulin constant region, such as a human IgG constant region, optionally a human IgG1 constant region. In some embodiments, the IgG1 constant region comprises one or more modifications such as LALA mutations.

In some embodiments, the antibody or antigen-binding portion thereof is a chimeric antibody or a humanized antibody.

In some embodiments, the antibody or antigen-binding portion thereof comprises:
(a) the amino acid sequence of SEQ ID No: 12 for the heavy chain and the amino acid sequence of SEQ ID No: 13 for the light chain; or
(b) the amino acid sequence of SEQ ID No: 14 for the heavy chain and the amino acid sequence of SEQ ID No: 15 for the light chain.

In one aspect, the disclosure provides an isolated nucleic acid molecule, comprising a nucleic acid sequence encoding the antibody or antigen-binding portion thereof as defined above.

In one aspect, the disclosure provides a vector comprising the nucleic acid molecule as defined above.

In one aspect, the disclosure provides a host cell comprising the nucleic acid molecule or the vector as defined above.

In one aspect, the disclosure provides a pharmaceutical composition comprising the antibody or antigen-binding portion thereof as defined above and a pharmaceutically acceptable carrier.

In one aspect, the disclosure provides a method for producing the antibody or antigen-binding portion thereof as defined above, comprising the steps of:
culturing the host cell under suitable conditions for expression of the antibody or antigen-binding portion thereof; and
isolating the antibody or antigen-binding portion thereof from the host cell.

In one aspect, the disclosure provides a method for modulating a B7H3-related immune response in a subject, comprising administering to the subject the antibody or antigen-binding portion thereof or the pharmaceutical composition as defined above to the subject.

In one aspect, the disclosure provides a method for preventing or treating B7H3-related disorders in a subject, comprising administering an effective amount of the antibody or antigen-binding portion thereof or the pharmaceutical composition as defined above to the subject.

In some embodiments, the B7H3-related disorder is selected from cancers, autoimmune diseases and infectious diseases. Said cancer may be selected from breast cancer, neurological tumor, melanoma, lung cancer, head and neck cancer, colorectal cancer, pancreatic cancer, stomach cancer, kidney cancer, bladder cancer, prostate cancer, ovarian cancer, cervical cancer, glioblastoma, esophageal cancer, bladder cancer, renal cell carcinoma, endometrial cancer, skin cancer, testis cancer, thyroid cancer, urothelial cancer, lymphoma such as non-Hodgkin's lymphoma, chronic lymphocytic leukemia, diffuse large B-cell lymphoma, and multiple myeloma.

In some embodiments, the antibody or antigen-binding portion thereof is administered in combination with cell immunotherapy, a chemotherapeutic agent, a radiation therapy, a targeted therapy and or other agents for use in cancer immunotherapy.

In one aspect, the disclosure provides the antibody or antigen-binding portion thereof as defined above for use in treating or preventing B7H3-related disorders in a subject.

In one aspect, the disclosure provides use of the antibody or antigen-binding portion thereof as defined above in the manufacture of a medicament for treating or preventing B7H3-related disorders in a subject.

In one aspect, the disclosure provides a kit comprising the antibody or antigen-binding portion thereof as defined above.

The foregoing is a summary and thus contains, by necessity, simplifications, generalizations, and omissions of detail; consequently, those skilled in the art will appreciate that the summary is illustrative only and is not intended to be in any way limiting. This summary is not intended to identify key features or essential features of the claimed subject matter, nor is it intended to be used as an aid in determining the scope of the claimed subject matter.

### BRIEF DESCRIPTION OF FIGURES

FIG. 1 shows SDS-PAGE result of W301088-1.145.16-z3-p1-uIgG1KV320. M: PageRuler™ Unstained Protein Ladder; lane 1: W301088-1.145.16-z3-p1-uIgG1KV320, Non-reducing; lane 2: W301088-1.145.16-73-p1-uIgG1KV320, Reducing; Gel info: NuPAGE, Novex 4-12% Bis-Tris Gel.

FIG. 2 shows HLPC-SEC result of W301088-1.145.16-z3-p1-uIgG1KV320.

FIG. 3 shows DSF profiles of W301088-1.145.16-z3-p1-uIgG1KV320.

FIG. 4 shows HIC-HPLC profile of W301088-1.145.16-z3-p1-uIgG1KV320.

FIG. 5 shows DLS-kp profile of W301088-1.145.16-73-p1-uIgG1KV320.

FIG. 6 shows FACS binding result of antibodies on human B7H3-expressing cell MCF-7.

FIG. 7 shows FACS binding result of antibodies on cynomolgus monkey B7H3-transfected cell.

FIGS. 8A-8C show binding of W301088-1.145.16-xIgG1KV320 (A), W301088-1.145.16-z3-p1-uIgG1KV320 (B) and Enoblituzumab of MacroGenics (C) to human 4IgB7H3.

FIGS. 9A-9C show binding of W301088-1.145.16-xIgG1KV320 (A), W301088-1.145.16-7.3-p1-uIgG1KV320 (B) and Enoblituzumab of MacroGenics (C) to human 2IgB7H3.

FIGS. 10A-10C show binding of W301088-1.145.16-xIgG1KV320 (A), W301088-1.145.16-z3-p1-uIgG1KV320 (B) and Enoblituzumab of MacroGenics (C) to cynomolgus monkey B7H3.

FIG. 11 shows internalization on human B7H3-expressing cell MCF-7 by different antibodies.

FIGS. 12A-12B show the alignment of the VH and VL regions between W301088-1.145.16-xIgG1KV320 and W301088-1.145.16-z3-p1-uIgG1KV320.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a”, “an” and “the” include plural referents unless the context clearly dictates otherwise. Thus, for example, reference to “a protein” includes a plurality of proteins; reference to “a cell” includes mixtures of cells, and the like. In this application, the use of “or” means “and or” unless stated otherwise. Furthermore, the use of the term “comprising,” as well as other forms, such as “comprises” and “comprised,” is not limiting. In addition, ranges provided in the specification and appended claims include both end points and all points between the end points.

In the context of the present disclosure, the term “about” denotes an interval of accuracy that the person of ordinary skill will understand to still ensure the technical effect of the feature in question. The term typically indicates deviation from the indicated numerical value by ±5%, such as ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. Sec, e.g., Abbas et al., Cellular and Molecular Immunology, 6^{th} ed., W. B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art. Further, the contents of all references, patents and published patent applications cited throughout this application are incorporated herein in entirety by reference.

### Definitions

In order to better understand the disclosure, the definitions and explanations of the relevant terms are provided as follows.

The term “antibody” or “Ab” herein is used in the broadest sense, which encompasses various antibody structures, including polyclonal antibodies, monospecific and multispecific antibodies (e.g. bispecific antibodies). A native intact antibody generally is a Y-shaped tetrameric protein comprising two heavy (II) and two light (L) polypeptide chains held together by covalent disulfide bonds and non-covalent interactions. Light chains of an antibody may be classified into κ and λ light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{L}). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (VH and VL.) of each heavy/light chain pair forms antigen binding sites, respectively. The extent of the framework region and CDRs can be precisely identified using methodology known in the art, for example, by the Kabat definition, the definitions at Dr. Martin's website, the Chothia definition, the AbM definition, the EU definition, and the contact definition, all of which are well known in the art. Sec, e.g., Kabat, E. A., et al. (1991) Sequences of Proteins of Immunological Interest, Fifth Edition, U.S. Department of Health and Human Services, NIH Publication No. 91-3242; Martin A. “Antibody bioinformatics website of Dr. Andrew Martin's lab at UCL,” last updated on 31 Jul. 2018; Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63 (1): 78-85; and Almagro, J. Mol. Recognit. 17:132-143 (2004). See also hgmp.mrc.ac.uk and bioinf.org.uk/abs. Correspondence or alignments between numberings according to different definitions can for example be found at http://www.imgt.org/(see also Giudicelli V et al., IMGT, the international ImMunoGeneTics database. Nucleic Acids Res. (1997) 25:206-11; Lefranc M P et al. Unique database numbering system for immunogenetic analysis. Immunol Today (1997) 18:509; and Lefranc M P et al., IMGT unique numbering for immunoglobulin and T cell receptor variable domains and Ig superfamily V-like domains. Dev Comp Immunol. (2003) 27:55-77). Antibodies may be of different antibody isotypes, for example, IgG (e.g., IgG1, IgG2, IgG3 or IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. In certain embodiments, an antigen-binding fragment of an antibody may contain at least one variable domain covalently linked to at least one constant domain. The variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule.

The term “variable region” or “variable domain” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet still retains the capability of binding to an antigen or forming an antigen-binding site.

The term “constant region” as used herein refers to immunoglobulin constant regions, which comprise the CH1, CH2 and CH3 domains in the heavy chain (and optionally the hinge region), and the constant domain in the light chain. Antibody heavy and light chain constant regions are well known in the art, e.g., those provided in the IMGT database (www.imgt.org) or at www.vbasc2.org/vbstat.php., both of which are incorporated by reference herein. “Fc” with regard to an antibody refers to that portion of the antibody comprising the second (CH2) and third (CH3) constant regions of a first heavy chain bound to the second and third constant regions of a second heavy chain via disulfide bonding. The Fc region may also comprise part or whole of the hinge region. The Fc region of the antibody is responsible for various effector functions such as ADCC and CDC, but does not function in antigen binding. The capacity of antibodies to initiate and regulate effector functions through their Fc domain is a key component of their in vivo protective activity. Although the neutralizing activity of antibodies has been previously considered to be solely the outcome of Fab-antigen interactions, it has become apparent that their in vivo activity is highly dependent on interactions of the IgG Fc domain with its cognate receptors, Fcγ receptors (FcγRs), expressed on the surface of effector leukocytes.

The term “B7H3”, also known as CD276 antigen, refers to a type-1 transmembrane protein that is a member of the B7 family possessing an ectodomain composed of a single IgV-IgC domain pair. B7 family proteins contain extracellular IgV-like and IgC-like domains with a short cytoplasmic tail. B7H3 is an immune checkpoint molecule and is aberrantly overexpressed in many types of cancers. When referring to the amino acid sequence of B7H3 protein, it includes full-length B7H3 protein (such as human 4IgB7H3 protein or human 2IgB7H3 protein), or the extracellular domain of B7113 (B7H3 ECD) or fragment containing B7H3 ECD; fusion protein of B7H3 ECD. Exemplary sequences of B7H3 proteins can be found at Uniprot ID: Q5ZPR3 (human 4IgB7H3), Genebank Accession Numbers NP_001019907 (human), NP_001316557 (human), NP_001316558 (human), NP_079516 (human), and NP_598744 (mouse). Cynomolgus monkey B7H3 shares approximately 97% and 88% amino acid sequence identity with human and mouse B7H3 respectively.

The term “an antibody that binds B7H3” or “an anti-B7113 antibody” as used herein includes antibodies and antigen-binding fragments thereof that specifically recognize a B7H3 protein, as well as antibodies and antigen-binding fragments thereof that specifically bind a B7H3 protein. As used herein, the expression “anti-B7H13 antibody” includes both monovalent antibodies with a single specificity, as well as bispecific antibodies comprising a first antigen-binding site that binds B7H3 and a second antigen-binding site that binds a second (target) antigen.

The term “monoclonal antibody” or “mAb”, as used herein, refers to a preparation of antibody molecules of single molecular composition. A monoclonal antibody displays a single binding specificity and affinity for a particular epitope.

The term “chimeric antibody”, as used herein, refers to antibodies comprising or consisting of an antibody's original antigen-binding variable domains (e.g. murine) with the constant domains from a different species (e.g. human). Preferably the term “chimeric antibody”, as used herein, refers to antibodies consisting of an antibody's original antigen-binding variable domains (e.g. murine) with the constant domains from a different species (e.g. human).

The term “humanized antibody” is intended to refer to antibodies in which CDR sequences derived from the germline of another mammalian species, such as a rat/mouse, have been grafted onto human framework sequences. Additional framework region modifications may be made within the human framework sequences. For example, certain residues in the framework region may be back mutated to original sequence to maintain affinity.

The term “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. In one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “K_{D}” as used herein, is intended to refer to the dissociation constant of a particular antibody-antigen interaction, which is obtained from the ratio of k_{d} to kₐ (i.e., k_{d}/kₐ) and is expressed as a molar concentration (M). The term “kₐ” as used herein, is intended to refer to the association rate of a particular antibody-antigen interaction, whereas the term “k_{d}” as used herein, is intended to refer to the dissociation rate of a particular antibody-antigen interaction. K_{D} values for antibodies can be determined using methods well established in the art. A preferred method for determining the K_{D} of an antibody is by using surface plasmon resonance, preferably using a biosensor system such as a Biacore® system.

The term “high affinity” for an IgG antibody, as used herein, refers to the strength of the binding interaction between antigen and antibody. Various methods are established in the art for measuring affinity, such as surface plasmon resonance (SPR), FACS affinity test, FACS binding test, and ELISA binding. In some embodiments, the antibody as disclosed herein has a K_{D} of 1×10⁻⁹ M or less, more preferably 5×10⁻¹⁰ M or less, even more preferably 1×10⁻¹⁰ M or less, even more preferably 9×10⁻¹¹ M or less and even more preferably 8×10⁻¹¹ M or less for a target antigen, as determined by SPR.

The term “EC₅₀”, as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “isolated”, as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody”, as used herein, is intended to refer to an antibody that is substantially free of other antibodies having different antigenic specificities (e.g., an isolated antibody that specifically binds to a B7H3 protein is substantially free of antibodies that specifically bind antigens other than B7H3 protein). An isolated antibody that specifically binds a human B7H3 protein may, however, have cross-reactivity to other antigens, such as B7H3 proteins from other species. Moreover, an isolated antibody can be substantially free of other cellular material and or chemicals.

The term “vector”, as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell”.

The term “identity”, as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity”, as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection”, as used herein, refers to the process by which nucleic acids are introduced into eukaryoticcells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, human B7H3 gene may be transfected into 293F or CHO cells.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS”, as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017 Nov. 9). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” includes any human or non-human animal. In some embodiments the subject is preferably human.

The term “B7H3-related disorder”, as used herein, refers to any condition or disease that is caused by, exacerbated by, or otherwise linked to increased or decreased (generally increased) expression or activities of B7H3 (e.g. a human B7H3).

The term “cancer,” as used herein, refers to any tumor or a malignant cell growth, proliferation or metastasis-mediated, solid tumors and non-solid tumors such as leukemia.

The term “treatment”, “treating” or “treated”, as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampening or slowing the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of B7H3-related diseases or conditions, refers to an antibody or antigen-binding portion thereof in an amount or concentration effective to treat or prevent the said diseases or conditions.

The term “prevent”, “prevention” or “preventing”, as used herein, with reference to a certain disease condition in a mammal, refers to preventing or delaying the onset of the disease, or preventing the manifestation of clinical or subclinical symptoms thereof.

The term “pharmaceutically acceptable”, as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and or physiologically compatible with a subject and an active agent, which is well known in the art (see, e.g., Remington's Pharmaceutical Sciences. Edited by Gennaro AR, 19th ed. Pennsylvania: Mack Publishing Company, 1995), and includes, but is not limited to, pH adjuster, surfactant, adjuvant and ionic strength enhancer. For example, the pH adjuster may include, but is not limited to, phosphate buffer; the surfactant may include, but is not limited to, cationic, anionic, or non-ionic surfactant, e.g., Tween-80; the ionic strength enhancer may include, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

Antibodies that Specifically Bind to B7H3

The disclosure provides antibodies that bind specifically to B7H3, such as human B7H3, mouse B7H3, cyno B7H3 and the ECD domain thereof. The term “antibody” as used herein includes both full-length immunoglobulins and antibody portions thereof that bind to the same antigen. The antibodies can be e.g. a monoclonal, polyclonal, chimeric, humanized or single chain antibody. In some embodiments, the antibody portions are Fab fragments or F(ab′) 2 fragments. In some embodiments, the antibody portions retain the ability to specifically bind B7H3.

Recombinant anti-B7H3 antibodies, such as chimeric and humanized monoclonal antibodies, comprising both human and non-human portions, which can be made using standard recombinant DNA techniques, are within the scope of the disclosure. Such chimeric and humanized monoclonal antibodies can be produced by recombinant DNA techniques, such as the methods described in U.S. Pat. No. 7,112,421; Better et al. (1988) Science 240:1041-1043; or Liu et ai. (1987) Proc. Natl. Acad. Sci. USA 84:3439-3443. Humanized monoclonal antibodies that are further modified/optimized, for example via affinity maturation, back mutation and removal of post-translational modification sites, are also included in the disclosure.

Preferably, the antibodies or antigen-binding portion thereof, as disclosed herein could bind to human 4IgB7H3 with high affinity. The antibodies or antigen-binding portion thereof, as disclosed herein could bind to human 2IgB7H3 with much lower affinity. The antibodies or antigen-binding portion thereof, as disclosed herein could bind to human 4IgB7H13 with high affinity but bind to human 2IgB7H3 with much lower affinity.

The binding of an antibody of the disclosure to B7H3 can be assessed using one or more techniques well established in the art, for instance, ELISA or flow cytometry. In some embodiments, the antibody can be tested by a flow cytometry assay in which the antibody is reacted with a cell line that expresses human B7H3, such as MCF-7 cancer cells or CHOK1 cells that have been transfected to express B7H3 on their cell surface. Additionally or alternatively, the binding of the antibody, including the binding kinetics (e.g., K_{D} value) can be tested in BIAcore binding assays. In some other embodiments, the antibodies are tested by a ELISA in which the antibody is reacted with a soluble B7H3 protein.

The disclosure provides antibodies that bind to a human 4IgB7H13 protein with a K_{D} of 1×10⁻⁸ M or less, a K_{D} of 1×10⁻⁹ M or less, a K_{D} of 5×10⁻¹⁰ M or less, a K_{D} of 1×10⁻¹⁰ M or less, a K_{D} of 9×10⁻¹¹ M or less, a K_{D} of 8×10⁻¹¹ M or less, or a K_{D} of 7×10⁻¹¹ M or less, as measured by Surface Plasmon Resonance (SPR). The antibodies as disclosed herein could also bind to human 2IgB7H3 protein with a K_{D} of 5×10⁻⁹ M or more, a K_{D} of 1×10⁻⁸ M or more, a K_{D} of 2×10⁻⁸ M or more, a K_{D} of 3×10⁻⁸ M or more, or a K_{D} of 4×10⁻⁸ M or more, as measured by Surface Plasmon Resonance (SPR). In some embodiments, the antibodies herein bind to human 4IgB7H3 with a K_{D} value that is more than 500 folds smaller, more than 100 folds smaller or more than 50 folds smaller than that of binding to human 2IgB7H3. These K_{D} value for comparison may be measured by Surface Plasmon Resonance (SPR).

In some embodiments, the antibodies of the disclosure are capable to bind to a human or cyno B7H3 expressing cell lines with an EC₅₀ of less than 5 nM, less than 4 nM, less than 3 nM, or less than 2 nM, as determined by FACS.

### Antibody Variable Domain and CDRs

In some embodiments, the antibody or antigen-binding portion thereof is a chimeric antibody or a murine antibody that specifically binds to B7H3, preferably human 4IgB7H3. In some further embodiments, the antibody or antigen-binding portion thereof is a humanized antibody that specifically binds to B7H3, preferably human 4IgB7H3. In some still further embodiments, the humanized antibody or antigen-binding portion thereof comprises one or more back mutations in the framework regions. In some still further embodiments, the humanized antibody or antigen-binding portion thereof comprises one or more modifications at potential post-translational modification (PTM) sites, for example, to remove NG, NS and DG in CDRs, NXS and NXT (X can be any amino acid except for P) in the entire length.

In some embodiments, the antibody or antigen-binding portion thereof as disclosed herein comprises one or more heavy chain CDRs (HCDRs) selected from at least one of the groups consisting of:
(i) a HCDR1 comprising SEQ ID No: 1 or a HCDR1 that differs in amino acid sequence from SEQ ID No: 1 by an amino acid addition, deletion or substitution of not more than 2 amino acids (e.g. 2 amino acids or 1 amino acid);
(ii) a HCDR2 comprising SEQ ID No: 2 or a HCDR2 that differs in amino acid sequence from SEQ ID No: 2 by an amino acid addition, deletion or substitution of not more than 2 amino acids (e.g. 2 amino acids or 1 amino acid); and
(iii) a HCDR3 comprising SEQ ID No: 3 or a HCDR3 that differs in amino acid sequence from SEQ ID No: 3 by an amino acid addition, deletion or substitution of not more than 2 amino acids (e.g. 2 amino acids or 1 amino acid); and/or
one or more light chain CDRs (LCDRs) selected from at least one of the groups consisting of:
(i) a LCDR1 comprising SEQ ID No: 4, 7 or 18 or a LCDR1 that differs in amino acid sequence from SEQ ID No: 4, 7 or 18 by an amino acid addition, deletion or substitution of not more than 2 amino acids (e.g. 2 amino acids or 1 amino acid);
(ii) a LCDR2 comprising SEQ ID NO: 5 or a LCDR2 that differs in amino acid sequence from SEQ ID NO: 5 by an amino acid addition, deletion or substitution of not more than 2 amino acids (e.g. 2 amino acids or 1 amino acid); and
(iii) a LCDR3 comprising SEQ ID NO: 6 or a LCDR3 that differs in amino acid sequence from SEQ ID NO: 6 by an amino acid addition, deletion or substitution of not more than 2 amino acids (e.g. 2 amino acids or 1 amino acid).

In some embodiments, the amino acid substitution(s) in the CDRs are conservative substitutions. In some embodiments, the antibody or antigen-binding portion thereof as described above may comprise a HCDR1 comprising or consisting of SEQ ID NO: 1; a HCDR2 comprising or consisting of SEQ ID NO: 2; a HCDR3 comprising or consisting of SEQ ID NO: 3; a LCDR1 comprising or consisting of SEQ ID NO: 7; a LCDR2 comprising or consisting of SEQ ID NO: 5; and a LCDR3 comprising or consisting of SEQ ID NO: 6. Specifically, the antibody may further comprise one or more modifications in the CDRs to remove potential PTM sites. In some embodiments, the antibody comprises one or more modifications in LCDR1 compared to the LCDR1 as shown in SEQ ID NO: 7 (“KSSQSLLNSSNQKNYLA”) to remove “NS”, which is a potential PTM site in the sequence. In some preferable embodiments, the antibody comprises one substitution at position 8 or 9 of the amino acid sequence of LCDR1 compared to the LCDR1 as shown in SEQ ID NO: 7 (“KSSQSLLNSSNQKNYLA”). In some more preferable embodiments, the antibody comprises a N to Q substitution at position 8 of the amino acid sequence of LCDR1 (i.e. SEQ ID NO: 4, “KSSQSLLOSSNQKNYLA”). In some other embodiments, the antibody comprises a S to P substitution at position 9 of the amino acid sequence of LCDR1 (i.e. SEQ ID NO: 18, “KSSQSLLNPSNQKNYLA”). A person skilled in the art would appreciate that, other types of substitutions can be selected as long as the binding affinity to B7H3 is substantially retained.

In some embodiments, the antibody or antigen-binding portion thereof as disclosed herein may comprise a HCDR1 comprising or consisting of SEQ ID NO: 1; a HCDR2 comprising or consisting of SEQ ID NO: 2; a HCDR3 comprising or consisting of SEQ ID NO: 3; a LCDR1 comprising or consisting of SEQ ID NO: 4; a LCDR2 comprising or consisting of SEQ ID NO: 5; and a LCDR3 comprising or consisting of SEQ ID NO: 6.

In some embodiments, the antibody or antigen-binding portion thereof comprises a heavy chain variable region (VH) and a light chain variable region (VL), wherein the heavy chain variable region and light chain variable region comprise the HCDR1-3 and LCDR1-3 as described above, respectively.

In some embodiments, the heavy chain variable region of the antibody or antigen-binding portion thereof comprises:
(i) the amino acid sequence of SEQ ID No: 8 or 10; (ii) an amino acid sequence at least 85%, 90%, or 95% identical to SEQ ID No: 8 or 10; or (iii) an amino acid sequence with addition, deletion and/or substitution of one or more (e.g. 1, 2, 3, 4, 5, 6, 7, 8, 9, 10 or more) amino acids compared with SEQ ID No: 8 or 10.

In some embodiments the amino acid substitution(s) may be conservative substitutions.

In some embodiments, the light chain variable region of the antibody or antigen-binding portion thereof comprises:
(i) the amino acid sequence of SEQ ID No: 9 or 11; (ii) an amino acid sequence at least 85%, at least 90%, or at least 95% identical to SEQ ID No: 9 or 11; or (iii) an amino acid sequence with addition, deletion and/or substitution of one or more (e.g. 1, 2, 3, 4, 5, 6, 7, 8, 9, 10 or more) amino acids compared with SEQ ID No: 9 or 11.

In some embodiments the amino acid substitution(s) may be conservative substitutions.

In some embodiments, the antibody or antigen-binding portion thereof comprises a heavy chain variable region (VH) and a light chain variable region (VL), wherein the VH comprises:
(i) the amino acid sequence of SEQ ID No: 8 or 10; or (ii) an amino acid sequence with substitution of one or more (e.g. 1, 2, 3, 4, 5 or more) amino acids in the framework region(s) compared with SEQ ID No: 8 or 10;
and or the VL comprises:
(i) the amino acid sequence of SEQ ID No: 9 or 11; or (ii) an amino acid sequence with substitution of one or more (e.g. 1, 2, 3, 4, 5 or more) amino acids in the framework region(s) compared with SEQ ID No: 9 or 11.

In some embodiments the amino acid substitution(s) may be conservative substitutions.

In some embodiments, the antibody or antigen-binding portion thereof comprises the HCDR1, HCDR2 and HCDR3 of the VH region as shown in SEQ ID No: 8 or 10, and LCDR1, LCDR2 and LCDR3 of the VL region as shown in SEQ ID No: 9 or 11.

As will be appreciated by those in the art, the exact numbering and placement of the CDRs can be different among different numbering systems. However, it should be understood that the disclosure of a variable heavy and or variable light sequence includes the disclosure of the associated (inherent) CDRs. Accordingly, the disclosure of each variable heavy region is a disclosure of the heavy chain CDRs (e.g., HCDR1, HCDR2 and HCDR3) and the disclosure of each variable light region is a disclosure of the light chain CDRs (e.g., LCDR1, LCDR2 and LCDR3). A useful comparison of CDR numbering is as below, see Lafranc et al., Dev. Comp. Immunol. 27 (1): 55-77 (2003):

| | | | | | Kabat + | Dr. Martin's (see Martin, |
|---|---|---|---|---|---|---|
| CDR | Kabat | IMGT | Chothia | AbM | Chothia | 2018 supra for details) |
| HCDR1 | 31-35 | 27-38 | 26-32 | 26-35 | 26-35 | CXXX + HCDR1 + W |
| HCDR2 | 50-65 | 56-65 | 52-56 | 50-58 | 50-65 | LEWG + HCDR2 |
| HCDR3 | 95-102 | 105-117 | 95-102 | 95-102 | 95-102 | CAR + HCDR3 + WGXG |
| LCDR1 | 24-34 | 27-38 | 24-34 | 24-34 | 24-34 | C + LCDR1 + W |
| LCDR2 | 50-56 | 56-65 | 50-56 | 50-56 | 50-56 | 16 residues after LCDR1 |
| LCDR3 | 89-97 | 105-117 | 89-97 | 89-97 | 89-97 | C + LCDR3 + FGXG |

The assignment of amino acids to each CDR may be in accordance with one or a combination of the numbering schemes provided above, all of which are well known in the art. See Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Martin A. “Antibody bioinformatics website of Dr. Andrew Martin's lab at UCL,” last updated on 31 Jul. 2018; Chothia et al., 1989, PMID: 2687698; MacCallum et al., 1996, PMID: 8876650; Dubel, Ed. (2007) Handbook of Therapeutic Antibodies, 3^{rd} Ed., Wily-VCH Verlag GmbH and Co; and http://www.imgt.org/.

Variable regions and CDRs in an antibody sequence can be identified according to general rules that have been developed in the art (as set out above, such as the Kabat, Martin and IMGT numbering system) or by aligning the sequences against a database of known variable regions. Methods for identifying these regions are described in Kontermann and Dubel, eds., Antibody Engineering, Springer, New York, NY, 2001 and Dinarello et al., Current Protocols in Immunology, John Wiley and Sons Inc., Hoboken, NJ, 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). For example, sequences may be analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13:978-3540413547, also available on the website bioinforg.uk/abs). The Abysis database website further includes general rules that have been developed for identifying CDRs which can be used in accordance with the teachings herein. Two antibodies having the same VH and or VL CDRs means that their CDRs are identical when determined by the same approach (e.g., the Kabat approach, Dr Martin's approach, the Chothia approach, or the IMGT numbering as known in the art).

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25 (17): 3389-3402. When utilizing BLAST and Gapped BLAST programs, the default parameters of the respective programs {e.g., XBLAST and NBLAST) can be used. See www.ncbi.nlm.nih.gov.

In other embodiments, the CDR amino acid sequences can be at least 80%, 81%, 82%, 83%, 84%, 85%, 86%, 87%, 88%, 89%, 90%, 91%, 92%, 93%, 94%, 95%, 96%, 97%, 98% or 99% identical to the respective sequences set forth above. In other embodiments, the amino acid sequences of the variable region can be at least 90%, 91%, 92%, 93%, 94%, 95%, 96%, 97%, 98% or 99% identical to the respective sequences set forth above.

As mentioned above, the addition, deletion and/or substitution of at least one of the amino acids in the VI or VL region may be not in any of the CDR sequences, but in the framework (FRW) sequences. For example, the isolated antibody or antigen-binding portion thereof may comprise one or more substitutions of the amino acids in the framework sequences, e.g. FRW1, FRW2, FRW3, and/or FRW4 of the VII or VL region. In some embodiments, the framework regions of the VII region comprise one or more of the following substitutions: S025T, V071R, Y091F, V089T.

In some embodiments, the set of 6 CDRs can have from 0, 1, 2, 3, 4 or 5 amino acid modifications (preferably amino acid substitutions) in total, as well as changes in the framework regions of the variable heavy and light regions, as long as the frameworks (excluding the CDRs) retain at least about 80%, 85% or 90% identity to a parental antibody (e.g. W301088-1.145.16). Thus, for example, the identical CDRs as described herein can be combined with different framework sequences from human germline sequences, as long as the framework regions retain at least 80%, 85% or 90% identity to a human germline sequence.

In certain embodiments, the isolated antibody or antigen-binding portion thereof as provided herein comprise any suitable framework region (FR) sequences, as long as the antigen-binding domains can specifically bind to B7H3, preferably human Ig4B7H3.

The framework regions of the isolated antibody or the antigen-binding portion thereof may contain one or more back mutations, wherein the framework amino acid(s) from human germlines are replaced by original murine amino acids at the corresponding place. The framework regions of the isolated antibody or the antigen-binding portion thereof may also comprise one or more conservative substitutions. The term “conservative substitution”, as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32:1180-1187 (1993); Kobayashi et al., Protein Eng. 12 (10): 879-884 (1999); and Burks et al., Proc. Natl. Acad. Sci. USA 94:412-417 (1997), which are incorporated herein by reference).

### IgG Constant Region

In some embodiments, the antibody further comprises an immunoglobulin constant region, such as a human IgG, IgA, IgM, IgE or IgD) constant region. The antibody as disclosed herein can be of various IgG isotype, e.g. IgG1, IgG2, IgG3 and IgG4. Preferably, the antibody is IgG1 isotype and comprises a human IgG1 constant region. The IgG constant region as described herein comprises the Fc region and optionally part or whole of the hinge region of the IgG molecule.

The Fc region may comprise one or more amino acid changes (e.g., insertions, deletions or substitutions) as compared to wild-type Fc region, without changing the desired functionality. For example, the disclosure includes antibodies comprising one or more modifications in the Fc region that may have a modified binding interaction (e.g., enhanced or diminished) between Fc and FcRn. Non-limiting examples of Fc modifications also include, e.g., an amino acid modification of Leu234Ala Leu235Ala (or LALA) in IgG1 Fc region that alters the antibody-dependent cellular cytotoxicity (ADCC) or other effector functions, a mutation of serine (“S”) to proline (“P”) at position 228 of the amino acid sequence of human IgG4 Fc region. The S228P mutation reduces Fab-arm exchange by stabilizing the disulfides in the core-hinge of the IgG4 molecules, thus belongs to an IgG4 stabilization mutation which helps prevent half-antibody formation. It is known that increased binding to FcγRIIIa generally results in increased ADCC. Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIGS. 41) and 8,084,582, and US Publ. application Nos. 20060235208 and 20070148170, all of which are expressly incorporated herein by reference in their entirety. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the antibodies as disclosed herein.

The variable regions are operably linked to the IgG constant region in the heavy chain and light chain, respectively. In some embodiments, the variable region is directly linked to the IgG constant region. In some other embodiments, the variable region is linked to the IgG constant region via a linker, which may comprise as few as one amino acid. Peptide linkers for constructing polypeptide sequences are well established in the art, such as (GS)n, (GSGGS)n, (GGGGS)n, and (GGGS) n linkers where n is an integer of at least one (and generally from 3 to 4 to 5), or linkers derived from immunoglobulin heavy chain or light chain (such as hinge region-derived sequences), as well as any peptide sequence that allows for recombinant attachment of the variable region to the constant region with sufficient length and flexibility to allow each region to perform its biological function.

Unless specified otherwise, the numbering of the constant region of the antibody herein is based on the EU numbering as in Kabat. Accordingly, “CH” domains in the context of IgG are as follows: “CH1” refers to positions 118-215 according to the EU index as in Kabat, “Hinge” refers to positions 216-230 according to the EU index as in Kabat, “CH2” refers to positions 231-340 according to the EU index as in Kabat, and “CH3” refers to positions 341-447 according to the EU index as in Kabat.

### Functionality of the Anti-B7H3 Antibodies

The antibodies or antigen-binding portions thereof as disclosed herein are characterized by particular functional features or properties. In some embodiments, the antibodies (including both the chimeric antibodies and humanized antibodies) have one or more of the following properties:
(a) specifically bind to human 4IgB7H3 expressing cells with a high affinity (e.g. less than 2 nM as measured by FACS);
(b) bind to human 2IgB7H13 with a significantly lower affinity compared to that of human 4IgB7H3, the discrepancy between antibody binding to 2IgB7H3 and to 4IgB7H3 are significant;
(c) specifically bind to cyno B7H3 with a high affinity (e.g. less than 5 nM as measured by FACS), better than the benchmark antibodies; and
(d) show good internalization ability to B7H3-expressing cancer cells.

### Nucleic Acid Molecules Encoding Antibodies of the Disclosure

In some aspects, the present disclosure is directed to an isolated nucleic acid molecule, comprising a nucleic acid sequence encoding the heavy chain variable region and or the light chain variable region of the isolated antibody as disclosed herein. In some embodiments, the isolated nucleic acid molecule disclosed herein comprises the nucleic acid sequence as set forth in SEQ ID No: 16. In some embodiments, the isolated nucleic acid molecule disclosed herein comprises the nucleic acid sequence as set forth in SEQ ID No: 17.

Nucleic acids of the present disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

The isolated nucleic acid encoding the VH region can be converted to a full-length heavy chain gene by operatively linking the VII-encoding nucleic acid to another DNA molecule encoding heavy chain constant regions (CH1, CH2 and CH3). The sequences of human heavy chain constant region genes are known in the art (see e.g., Kabat et al. (1991), supra) and DNA fragments encompassing these regions can be obtained by standard PCR amplification. The heavy chain constant region can be an IgG1, IgG2, IgG3, IgG4, IgA, IgE, IgM or IgD constant region, but more preferably is an IgG1 or IgG4 constant region.

The isolated nucleic acid encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat et al., supra) and DNA fragments encompassing these regions can be obtained by standard PCR amplification. In preferred embodiments, the light chain constant region can be a kappa or lambda constant region.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

In some aspects, the disclosure is directed to a vector comprising the nucleic acid sequence as disclosed herein. A vector in the context of the present disclosure may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In some embodiments, a B7H3 antibody-encoding nucleic acid is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaP04-precipitated construct (as described in for instance WO200046147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the antibody in a bacterial cell. Examples of such vectors include expression vectors such as BlueScript (Stratagene), pIN vectors (Van Hecke& Schuster, J Biol Chem 264, 5503-5509 (1989), pET vectors (Novagen, Madison WI) and the like). A vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

A vector may also or alternatively be a vector suitable for expression in mammalian cells, e.g. a vector comprising glutamine synthetase as a selectable marker, such as the vectors described in Bebbington (1992) Biotechnology (NY) 10:169-175.

A nucleic acid and or vector may also comprise a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The vector may comprise or be associated with any suitable promoter, enhancer, and other expression-facilitating elements. Examples of such elements include strong expression promoters (e. g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In some aspects, the disclosure relates to a host cell comprising the vector specified herein above.

Thus, the present disclosure also relates to a recombinant eukaryotic or prokaryotic host cell which produces the antibody of the present disclosure, such as a transfectoma. The antibody may be expressed in a recombinant eukaryotic or prokaryotic host cell to produce the antibody of the disclosure.

Examples of host cells include yeast, bacterial, plant and mammalian cells. Mammalian host cells for expressing the antibodies of the present disclosure include Chinese Hamster Ovary (CHO cells) (including dhfr CHO cells, described in Urlaub and Chasin, (1980) Proc. Natl. Acad. ScL USA 77:4216-4220, used with a DHFR selectable marker, e.g., as described in R. J. Kaufman and P. A. Sharp (1982) J. Mol. Biol. 159:601-621), COS cells and SP2 cells. In particular, for use with NSO myeloma cells, another expression system is the GS gene expression system disclosed in WO 87/04462, WO 89/01036 and EP 338,841. Also included are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., 1980, Proc. Natl. Acad. Sci. USA 77:4216); mouse sertoli cells (TM4, Mather, 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; mouse myeloma cells, such as NSO (e.g. RCB0213, 1992, Bio/Technology 10:169) and SP2/0 cells (e.g. SP2/0-Ag14 cells, ATCC CRL 1581); rat myeloma cells, such as YB2/0 cells (e.g. YB2/3HL.P2.G11.16Ag.20 cells, ATCC CRL 1662); PER.C6 cells; and a human hepatoma line (Hep G2). CHO cells are one of the cell lines that can be used herein, with CHO-K1, DUK-B11, CHO-DP12, CHO-DG44 (Somatic Cell and Molecular Genetics 12:555 (1986)), and Lec13 being exemplary host cell lines. In the case of CHO-K1, DUK-B11, DG44 or CHO-DP12 host cells, these may be altered such that they are deficient in their ability to fucosylate proteins expressed therein. In some embodiments, the host cells herein are selected from CHO, CHO-S, HEK, HEK293, HEK-293F, Expi293F, PER.C6 or NSO cells or lymphocytic cells.

Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for bispecific antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. buIgaricus (ATCC 16,045), K. wickcramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma recsia (EP 244,234); Neurosporacrassa; Schwanniomyces such as Schwanniomycesoccidentalis; and filamentous fungi such as, e.g., Neurospora, Penicillium, Tolypocladium, and Aspergillus hosts such as A. nidulans and A. niger.

In some embodiments, the host cell may comprise a first and second nucleic acid construct stably integrated into the cellular genome. In another embodiment, the present disclosure provides a cell comprising a non-integrated nucleic acid, such as a plasmid, cosmid, phagemid, or linear expression element, which comprises a first and second nucleic acid construct as specified above.

In an even further aspect, the disclosure relates to a transgenic non-human animal or plant comprising nucleic acids encoding one or two sets of a human heavy chain and a human light chain, wherein the animal or plant produces the antibody of the disclosure.

In a further aspect, the disclosure relates to a hybridoma which produces an antibody of the disclosure as defined herein. In an even further aspect, the disclosure relates to a transgenic non-human animal or plant comprising nucleic acids encoding one or two sets of a human heavy chain and a human light chain, wherein the animal or plant produces an antibody of the disclosure.

In one aspect, the disclosure relates to an expression vector comprising:
(i) a nucleic acid sequence encoding the antibody or antigen-binding portion thereof, or the VH or VL domain of the antibody according to any one of the embodiments disclosed herein;
(ii) a nucleic acid sequence encoding the amino acid sequence of a heavy chain or a light chain;
(iii) a nucleic acid sequence encoding the amino acid sequence of the constant region; or
(iv) the combinations of two or more of the above.

In one aspect, the disclosure relates to a nucleic acid construct encoding one or more amino acid sequences set out in the sequence listing.

In one aspect, the disclosure relates to a method for producing an antibody according to any one of the embodiments as disclosed herein, comprising the steps of culturing a host cell comprising an expression vector or more than one expression vectors as disclosed herein, expressing the antibody and purifying said antibody from the culture media. In one aspect, the disclosure relates to a host cell comprising an expression vector as defined above. In one embodiment, the host cell is a recombinant eukaryotic, recombinant prokaryotic, or recombinant microbial host cell.

### Pharmaceutical Compositions

In some aspects, the present disclosure is directed to a pharmaceutical composition comprising at least one antibody or antigen-binding portion thereof as disclosed herein and a pharmaceutically acceptable carrier.

### Components of the Compositions

The pharmaceutical composition may optionally contain one or more additional pharmaceutically active ingredients, such as another antibody or a drug. The pharmaceutical compositions of the present disclosure also can be administered in a combination therapy with, for example, another immune-stimulatory agent, anti-cancer agent, an antiviral agent, or a vaccine, such that an additional or synergistic effect may be achieved. A pharmaceutically acceptable carrier can include, for example, a pharmaceutically acceptable liquid, gel or solid carrier, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, as well as various combinations of components known in the art.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. According to the present disclosure, a solvent containing an antibody or an antigen-binding fragment of the present disclosure may include one or more anti-oxidants such as methionine, reducing antibody or antigen binding fragment thereof may be oxidized. The oxidation reduction may prevent or reduce a decrease in binding affinity, thereby enhancing antibody stability and extending shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increase activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the antibody or the antigen binding portion thereof of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight per dose.

In any event, the antibody or the antigen binding portion thereof of the disclosure is preferably administered as needed to a subject in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody or the antigen-binding portion thereof of the instant disclosure will comprise multiple doses of the selected drug product over a period of weeks or months. More specifically, the antibody or the antigen-binding portion thereof of the instant disclosure may be administered once every day, every two days, every four days, every week, every ten days, every two weeks, every three weeks, every month, every six weeks, every two months, every ten weeks, every three months or less frequently. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the antibody or antigen-binding portion thereof as disclosed herein in concentrations of from about 10 μg/mL to about 100 mg/mL. In certain selected embodiments, the concentrations of the antibody or the antigen binding portion thereof will comprise about 20 μg/mL, 40 μg/mL, 60 μg/mL, 80 μg/mL, 100 μg/mL, 200 μg/mL, 300 μg/mL, 400 μg/mL, 500 μg/mL, 600 μg/mL, 700 μg/mL, 800 μg/mL, 900 μg/mL or 1 mg/mL. In other preferred embodiments, the concentrations of the antibody or the antigen binding portion thereof will comprise about 2 mg/mL, 3 mg/mL, 4 mg/mL, 5 mg/mL, 6 mg/mL, 8 mg/mL, 10 mg/mL, 12 mg/mL, 14 mg/mL, 16 mg/mL, 18 mg/mL, 20 mg/mL, 25 mg/mL, 30 mg/mL, 35 mg/mL, 40 mg/mL, 45 mg/mL, 50 mg/mL, 60 mg/mL, 70 mg/mL, 80 mg/mL, 90 mg/mL or 100 mg/mL.

### Applications of the Disclosure

In some aspects, the present disclosure provides a method of preventing or treating a B7H3-related disease, disorder or condition in a subject, which comprises administering to the subject (for example, a human) in need of treatment a therapeutically effective amount of the antibody as disclosed herein. The disease or condition may be a cancer, an autoimmune disease or an infectious disease.

A variety of cancers where B7H3 is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, chondromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In some specific embodiments, the cancer is selected from neurological tumor, melanoma, lung cancer, head and neck cancer, colorectal cancer, pancreatic cancer, stomach cancer, kidney cancer, bladder cancer, prostate cancer, breast cancer and ovarian cancer.

In some embodiments, examples of cancer include but are not limited to B-cell cancers, including B-cell lymphoma (including low grade follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and or lymphoma, small lymphocytic lymphoma, B-cell chronic lymphocytic leukemia and/or prolymphocytic leukemia and or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade follicular lymphoma, intermediate grade follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and or Sezary syndrome, skin (cutaneous) lymphomas, anaplastic large cell lymphoma, angiocentric lymphoma.

In some embodiments, examples of cancer include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and or j) Hodgkin's disease.

In some other embodiments, the disease or condition is an autoimmune disease. Examples of autoimmune diseases that may be treated with the antibody or antigen-binding portion thereof as disclosed herein include autoimmune encephalomyelitis, lupus erythematosus, and rheumatoid arthritis, among others. The antibody or the antigen-binding portion thereof may also be used to treat or prevent infectious disease, inflammatory disease (such as allergic asthma) and chronic graft-versus-host disease.

### Combined Use

The antibody may be used in combination with a different anti-cancer agent, a cytotoxic agent, a chemotherapeutic agent, or a cell immunotherapy. For example, the antibodies of the disclosure may be administered in conjunction with an anti-PD-1/PD-L1 antibody. The antibodies of the disclosure may also be administered in conjunction with CAR-T therapy.

### Cellular Immunotherapies

In some embodiments, the antibody as disclosed herein may be used in combination with a cellular immunotherapy, also known as adoptive cell therapy. As is generally known, cellular immunotherapy is a form of treatment that uses the cells of human body's immune system to eliminate cancer. Some of these approaches involve directly isolating our own immune cells and simply expanding their numbers (e.g. performed by activating and expanding the immune cells of patient outside of the body and infused into the patient), whereas others involve genetically engineering immune cells (via gene therapy) to enhance their cancer-fighting capabilities. Cellular immunotherapies can be deployed in different ways, including but not limited to Tumor-Infiltrating Lymphocyte (TIL) therapy, Engineered T Cell Receptor (TCR-T) therapy, Chimeric Antigen Receptor (CAR) T Cell therapy, CAR NK Cell therapy, Natural Killer (NK) Cell therapy.

In some embodiments, the antibody may be used in combination with an additional anti-tumor therapy, such as tumor-infiltrating lymphocyte (TIL) therapy, T cell receptor T cell (TCR-T) therapy, chimeric antigen receptor (CAR) T cell therapy, and NK cell therapy, as well as targeted therapy and chemotherapy. The administration of the additional anti-tumor therapy may be performed before, after or simultaneously with the administration of the antibody or the pharmaceutical composition as disclosed herein.

### Chemotherapies

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in certain embodiments selected anti-cancer agents will be linked to the unpaired cysteines of the engineered antibodies to provide engineered conjugates as set forth herein. Accordingly, such engineered conjugates are expressly contemplated as being within the scope of the instant disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleuritesfordii proteins, dianthin proteins, Phytolaccamericana proteins (PAPI, PAPII, and PAP-S), Momordica charantia inhibitor, curcin, crotin, chlorambu officinalis inhibitor, gelonin, mitegellin, restrictocin, phenomycin, neomycin, and the tricothecenes) or animals, (e.g., cytotoxic Rnases, such as extracellular pancreatic Rnases; Dnase I, including fragments and/or variants thereof).

For the purposes of the instant disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the site-specific constructs of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSKR polysaccharide complex (JIIS Natural Products, Eugene, OR), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZARR gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX®rmRH; Vinorelbine and Esperamicins and pharmaceutically acceptable salts, acids or derivatives of any of the above.

Combined Use with Radiotherapies

The present disclosure also provides for the combination of the antibody with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed antibodies may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### Pharmaceutical Packs and Kits

Pharmaceutical packs and kits comprising one or more containers, comprising one or more doses of the antibody or the antigen-binding portion thereof are also provided. In certain embodiments, a unit dosage is provided wherein the unit dosage contains a predetermined amount of a composition comprising, for example, the antibody or the antigen-binding portion thereof, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and or be formulated within a stable and effective pH range. Alternatively, in certain embodiments, the composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed composition is used for treating the neoplastic disease condition of choice.

The present disclosure also provides kits for producing single-dose or multi-dose administration units of antibodies and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed antibodies or antigen-binding portion thereof. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the antibodies and, optionally, one or more anti-cancer agents in the same or different containers. The kits may also contain other pharmaceutically acceptable formulations, either for diagnosis or combined therapy. For example, in addition to the antibody or the antigen-binding portion thereof of the present disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed antibody or the antigen-binding portion thereof, with or without additional components, or they may have distinct containers for each desired agent. Alternatively, the antibodies and any optional anti-cancer agent of the kit may be maintained separately within distinct containers prior to administration to a patient. The kits may also comprise a second/third container means for containing a sterile, pharmaceutically acceptable buffer or other diluents such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody or the antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

Appended to the instant application is a sequence listing that comprises a number of amino acid sequences and nucleotide sequences. The following Tables A, B, and C provide a summary of the sequences of exemplified W301088 antibodies herein, including W301088-1.145.16, its chimeric version W301088-1.145.16-xIgG1KV320 and humanized version W301088-1.145.16-z3-p1-uIgG1KV320. W301088-1.145.16-xIgG1KV320 have the same variable regions as W301088-1.145.16 parental murine clone.

TABLE A
Amino acid sequences of W301088-1.145.16-xIgG1KV320 (i.e. the chimeric
antibody) with CDR parts underlined

| | |
|---|---|
| VH | DVQLQESGPGLVKPSQSLSLTCTVTDYSITGDYAWNWIRQFPGN |
| SEQ ID No: 8 | KLEWMGYISYSGSTSYNPSLQSRISITRDTSKNQFFLQLNSVTSED |
| | TATYFCARSLGRRWYFVVWGAGTTVTVSA |
| VL | DIVMTQSPSSLAMSVGQKVTMSCKSSQSLLNSSNQKNYLAWYQ |
| SEQ ID No: 9 | QKPGQSPKLLIYFASTRESGVPDRFIGSGSGTDFTLTISSVQAEDL |
| | TDYFCQQHYSAPWTFGGGTKLEIK |
| Heavy chain | DVQLQESGPGLVKPSQSLSLTCTVTDYSITGDYAWNWIRQFPGN |
| SEQ ID No: 14 | KLEWMGYISYSGSTSYNPSLQSRISITRDTSKNQFFLQLNSVTSED |
| | TATYFCARSLGRRWYFVVWGAGTTVTVSA |
| | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHKP |
| | SNTKVDKRVEPKSCDKTHTCPPCPAPEAAGGPSVFLFPPKPKDTL |
| | MISRTPEVTCVVVDVSHEDPEVKFNWYVDGVEVHNAKTKPREE |
| | QYNSTYRVVSVLTVLHQDWLNGKEYKCKVSNKALPAPIEKTIS |
| | KAKGQPREPQVYTLPPSREEMTKNQVSLTCLVKGFYPSDIAVEW |
| | ESNGQPENNYKTTPPVLDSDGSFFLYSKLTVDKSRWQQGNVFSC |
| | SVMHEALHNHYTQKSLSLSPG |
| Light chain | DIVMTQSPSSLAMSVGQKVTMSCKSSQSLLNSSNQKNYLAWYQ |
| SEQ ID No: 15 | QKPGQSPKLLIYFASTRESGVPDRFIGSGSGTDFTLTISSVQAEDL |
| | TDYFCQQHYSAPWTFGGGTKLEIK |
| | RTVAAPSVFIFPPSDEQLKSGTASVVCLLNNFYPREAKVQWKVD |
| | NALQSGNSQESVTEQDSKDSTYSLSSTLTLSKADYEKHKVYACE |
| | VTHQGLSSPVTKSFNRGEC |

TABLE B
Amino acid sequences of W301088-1.145.16-z3-p1-uIgG1KV320 (i.e. the final
lead antibody) with CDR parts underlined

| | |
|---|---|
| VH | QVQLQESGPGLVKPSQTLSLTCTVTDYSITGDYAWNWIRQHPGK |
| SEQ ID No: 10 | GLEWIGYISYSGSTSYNPSLQSRVTISRDTSKNQFSLKLSSVTAAD |
| | TAVYFCARSLGRRWYFVVWGQGTTVTVSS |
| VL | DIVMTQSPDSLAVSLGERATINCKSSQSLLQSSNQKNYLAWYQQ |
| SEQ ID No: 11 | KPGQPPKLLIYFASTRESGVPDRFSGSGSGTDFTLTISSLQAEDVA |
| | VYYCQQHYSAPWTFGGGTKVEIK |
| Heavy Chain | QVQLQESGPGLVKPSQTLSLTCTVTDYSITGDYAWNWIRQHPGK |
| SEQ ID No: 12 | GLEWIGYISYSGSTSYNPSLQSRVTISRDTSKNQFSLKLSSVTAAD |
| | TAVYFCARSLGRRWYFVVWGQGTTVTVSSASTKGPSVFPLAPSS |
| | KSTSGGTAALGCLVKDYFPEPVTVSWNSGALTSGVHTFPAVLQS |
| | SGLYSLSSVVTVPSSSLGTQTYICNVNHKPSNTKVDKRVEPKSCD |
| | KTHTCPPCPAPEAAGGPSVFLFPPKPKDTLMISRTPEVTCVVVDV |
| | SHEDPEVKFNWYVDGVEVHNAKTKPREEQYNSTYRVVSVLTVL |
| | HQDWLNGKEYKCKVSNKALPAPIEKTISKAKGQPREPQVYTLPP |
| | SREEMTKNQVSLTCLVKGFYPSDIAVEWESNGQPENNYKTTPPV |
| | LDSDGSFFLYSKLTVDKSRWQQGNVFSCSVMHEALHNHYTQKS |
| | LSLSPGK |
| Light Chain | DIVMTQSPDSLAVSLGERATINCKSSQSLLQSSNQKNYLAWYQQ |
| SEQ ID No: 13 | KPGQPPKLLIYFASTRESGVPDRFSGSGSGTDFTLTISSLQAEDVA |
| | VYYCQQHYSAPWTFGGGTKVEIKRTVAAPSVFIFPPSDEQLKSG |
| | TASVVCLLNNFYPREAKVQWKVDNALQSGNSQESVTEQDSKDS |
| | TYSLSSTLTLSKADYEKHKVYACEVTHQGLSSPVTKSFNRGEC |

TABLE C
Nucleotide sequences encoding VH and VL regions of W301088-1.145.16-z3-
p1-uIgG1KV320

| | |
|---|---|
| VH | CAGGTGCAGCTCCAGGAGTCTGGCCCTGGCCTGGTGAAGCCATCTCAGAC |
| SEQ ID No: 16 | ACTGAGCCTGACATGTACCGTGACCGATTATAGCATCACCGGCGACTACGC |
| | TTGGAATTGGATCAGACAGCACCCTGGAAAGGGCCTGGAGTGGATTGGAT |
| | ACATCAGCTATTCTGGCAGCACATCTTATAATCCTAGCCTGCAGTCTAGGG |
| | TGACAATTTCAAGAGATACCAGCAAAAATCAGTTTAGCCTGAAACTGAGC |
| | TCTGTGACCGCCGCCGATACAGCCGTGTATTTTTGTGCCAGAAGCCTGGGA |
| | AGAAGATGGTATTTTGTGGTGTGGGGACAGGGCACAACAGTGACCGTGTC |
| | TAGC |
| VL | GATATTGTGATGACCCAGTCTCCTGATAGCCTGGCCGTGAGCCTGGGCGAG |
| SEQ ID No: 17 | AGAGCCACAATCAATTGTAAGTCTAGCCAGTCTCTGCTGCAGAGCTCTAAT |
| | CAGAAAAATTACCTGGCCTGGTATCAGCAGAAGCCTGGCCAGCCTCCTAA |
| | GCTGCTGATCTACTTTGCCAGCACAAGGGAATCTGGAGTGCCTGATAGGTT |
| | TAGCGGCAGCGGCTCTGGCACAGATTTTACACTGACAATCTCTAGCCTGCA |
| | GGCCGAGGATGTGGCCGTGTATTATTGTCAGCAGCACTATTCTGCCCCTTG |
| | GACATTTGGCGGCGGCACCAAAGTGGAGATTAAG |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Antigens, Benchmark Antibodies and Cell Lines

### 1.1 Preparation of Commercial Materials

TABLE 1
Information of commercially available materials

| Materials | Vendor | Cat. |
|---|---|---|
| MCF7 | ATCC | HTB-22 |
| CHOK1 | ATCC | CTL-61 |
| Expi293 ™ Expression System Kit | Invitrogen | A14635 |
| Expi293F ™ expression medium | Invitrogen | A1435101 |
| Flp-In ™-CHO Cell Line | Invitrogen | R75807 |
| TMB Single Solution | Invitrogen | 002023 |
| Lipofectamine ™ 2000 Transfection | Invitrogen | 11668027 |
| Reagent | | |
| FreeStyle ™ CHO Expression Medium | Gibco | 12651014 |
| Hoechst 33342 | Invitrogen | H3570 |
| Calcein AM | Invitrogen | C3099 |
| pHrodo iFL Red STP | Invitrogen | P36011 |
| NuPAGE4%-12% Bis-Tris Gel | Invitrogen | NP0322BOX |
| 10 × PBS | Invitrogen | AM9624 |
| RPMI Medium 1640 (1X) | Gibco | 22400089 |
| 0.25% Trypsin-EDTA (1x) | Gibco | 25200072 |
| FBS | ExCell Bio | FND500 |
| 4% PFA | DING GUO | AR-0211 |
| BSA | BOVOGEN | BSAS 1.0 |
| DPBS | CORNING | 21-031-CVC |
| Ni-column | Cytiva | 173712 |
| Protein A column | Cytiva | 175438 |
| TSKgel G3000SWXL column | Tosoh | 0008541 |
| Goat anti-human-IgG-Fc-HRP | Bethyl | A80-304P |
| Goat anti-Mouse IgG-Fc-HRP | Bethyl | A90-231P |
| Goat anti-Mouse IgG1 | Bethyl | A90-205A |
| Goat anti-Mouse IgG2a | Bethyl | A90-207A |
| Goat anti-Mouse IgG2b | Bethyl | A90-209A |
| Goat anti-Mouse IgG3 | Bethyl | A90-211A |
| goat anti-mouse IgG-Fc Alexa 647 | Jackson ImmunoResearch | 115-605-164 |
| Alexa 647 goat anti-human antibody | Jackson ImmunoResearch | 109-605-098 |
| goat anti-mouse IgG Fc PE | Jackson ImmunoResearch | 115-115-164 |
| goat anti-human IgG Fc PE | Jackson ImmunoResearch | 109-115-098 |
| Anti-human Fc IgG | Jackson ImmunoResearch | 109-005-098 |
| Anti-mouse Fc IgG | SouthernBiotech | 1013-01 |
| Goat Anti-Mouse kappa light chain-HRP | Southern Biotech | A90-119P |
| Goat Anti-Mouse lambda light chain- | Southern Biotech | A90-121P |
| HRP | | |
| F(ab′)2-Goat anti-human-IgG-Fc | Jackson ImmunoResearch | 109-006-098 |
| F(ab′)2-Goat anti-mouse-IgG-Fc | Jackson ImmunoResearch | 115-005-064 |
| Human B7-H3/CD276 Protein, his tag | ACROBiosystems | B73-H52E2 |
| (2IgB7H3) | | |
| Cynomolgus B7-H3/CD276 Protein, | ACROBiosystems | B73-C52Ha |
| His Tag | | |
| Series S Sensor Chip CM5 | Cytiva | 29-1496-03 |
| Amine Coupling Kit | Cytiva | BR100050 |
| 10xHBS-EP+ | Cytiva | BR100669 |

### 1.2 Antigen Generation

The amino acid sequence encoding the extracellular domain of human 4IgB7H3 (Uniprot ID: Q5ZPR3, amino acids 29-461) was first codon optimized for mammalian expression and then synthesized by Sangon Biotech (Shanghai, China). The DNA segment was sub-cloned into the pcDNA3.3 expression vector with 6×His at the C-terminal, which was then transfected into Expi293F cells (Invitrogen, A14635). After five days of incubation, the supernatant was purified using Ni-column (Cytiva, 173712). Eluted protein was dialyzed into PBS via dialysis bag (Spectrum, 888-10987, MWCO 3.5 kDa). Protein concentration was determined by absorbance at 280 nm on a NanoDrop device. 2 μg of the purified protein was run on the SDS-PAGE gel (Invitrogen) with and without reducing reagent. The purity of the purified protein was quantified by HPLC-SEC with the TSKgel G3000SWXL size exclusion column (Tosoh, 008541). Purified protein was stored at −80° C. Human 2IgB7H3 and cynomolgus monkey B7H3 were purchased from ACRO (Cat. B73-H52E2) and ACRO (Cat. B73-C52Ha).

### 1.3 Production of Benchmark Antibodies (BMKs)

Anti-B7113 antibody Enoblituzumab (MacroGenics, US20120294796A1: Seq 117, 119) was used as benchmark antibody. The nucleic acid sequences encoding the variable domain of the antibody was first codon optimized for mammalian expression and then synthesized by Sangon Biotech (Shanghai, China). The DNA segments were then sub-cloned into modified pcDNA3.4 expression vectors with constant region of human IgG1. The plasmids containing VH and VL gene were co-transfected into Expi293F cells (Invitrogen, A14635). After five days of incubation, the supernatant was purified using Protein A column (Cytiva, 175438). Eluted protein was dialyzed into PBS via dialysis bag (Spectrum, 888-10987). Protein concentration was determined by absorbance at 280 nm on a NanoDrop device. 2 μg of the purified antibody was run on the SDS-PAGE gel (Invitrogen) with and without reducing reagent. The purity of the purified antibody was quantified by HPLC-SEC with the TSKgel G3000SWXL size exclusion column (Tosoh, 008541). Purified antibody was stored at −80° C.

### 1.4 Establishment of Stable Cell Lines/Cell Pool

Human 4IgB7H3-expressing cell lines were generated. Briefly, CHOK1 cells were transfected with pcDNA3.3 expression vector containing full-length of 4IgB7H3 using Lipofectamine 2000 transfection kit (Invitrogen, 11668027) according to manufacturer's protocol. At 48 h post transfection, the cells were subcultured to T75 flask in selective media (F12-K with 10% FBS and 15 μg/mL Blasticidin). After two or three passages of selection, Human 4IgB7H3 expression stable cell line (W3XX088-CHOK1.hPro1.2A5) was obtained by Blasticidin selection and limited dilution and the expression level was determined by FACS using anti-B7H3 antibody.

Cynomolgus monkey B7H3-expressing cell pools were generated. Briefly, FlipinCHO cells were transfected with pcDNA5/pOG44 expression vector containing full-length of cynomolgus monkey B7H3 using Lipofectamine 2000 transfection kit (Invitrogen, 11668027) according to manufacturer's protocol. At 48 h post transfection, the cells were subcultured to T75 flask in selective media (F12 with 10% FBS and 600 μg/mL Hygromycin B). After two or three passages of selection, high expression stable cell pool (WBP3XX088-FlpinCHO.cProl.pool) was obtained by Hygromycin B selection and BD FACSMelody™ cells sorting.

### Example 2

### Generation of WBP301088 Antibodies

### 2.1 Animal Immunization

4 Balb/c mice at age of 6-8 weeks were immunized with 200-400 μg human 4IgB7H3 encoding DNA plasmid/animal. The adjuvant mixture includes Adju-Phos, CpG-ODN and GM-CSF. The animals were injected once every other week via subcutaneous, intramuscular and hydrodynamic tail vein. The blood was collected from mice after 3 (1st-bleed) and 5 injections (2nd bleed). The serum titer was measured by ELISA and FACS.

ELISA assay was used to measure serum antibody titers against 4IgB7H3 antigen. Plates (Nunc) were coated with 100 μL of his tag at 0.5 μg/mL at 4° C. overnight, and then blocked with blocking buffer (2% BSA in PBS) for 1 hour at ambient temperature. The plates were then washed and incubated with 1 μg/mL antigen for 1 hour at ambient temperature. After washing, mouse serum was 1:3 serially diluted starting at 1:100 dilutions in blocking buffer and incubated for 2 hours at ambient temperature. The plates were then washed and subsequently incubated with secondary antibody goat anti-mouse IgG-Fc-HRP (Bethyl, A90-231P) for 1 hour. After washing, TMB substrate was added and the interaction was stopped by 2 M HCl. The absorbance at 450 nm was read using a microplate reader (Molecular Device). Serum titer was determined at 2-folds background.

TABLE 2
Summary of serum titer test on human 4IgB7H3 binding by ELISA

| Titer | mouse #1 | mouse #2 | mouse #3 | mouse #4 |
|---|---|---|---|---|
| 2nd-bleed | 218700 | 1968300 | 656100 | 1968300 |
| 1st-bleed | 218700 | 218700 | 656100 | 218700 |
| pre-bleed | <100 | <100 | <100 | <100 |

FACS assay was used to measure serum antibody titer against 4IgB7H3 antigen. Briefly, the engineered B7H3-expressing cells were seeded into 96-well U-bottom plates at a density of 1×10⁵ cells/well and centrifuged at 1500 rpm at 4° C. for 4 minutes before removing the supernatant. Mouse serum was 1:3 serially diluted starting at 1:100 dilutions in 1×PBS/1% BSA were added to re-suspend cells and incubated at 4° C. for 1 hour. The cells were washed twice with 180 μL 1×PBS/1% BSA. The secondary antibody, goat anti-mouse IgG-Fc Alexa 647 (Jackson, 109-605-098) was added to re-suspend cells and incubated at 4° C. in the dark for 30 minutes followed by washing with 180 μL 1×PBS/1% BSA. At the end, the cells were re-suspended in 100 μL 1×PBS/1% BSA, and the fluorescence intensity was measured by FACS (BD Canto II) and analyzed by FlowJo Version software. Serum titer was determined at 2-fold background.

TABLE 3
Summary of serum titer test on human 4IgB7H3 cell line by FACS

| Titer | mouse #1 | mouse #2 | mouse #3 | mouse #4 |
|---|---|---|---|---|
| 2nd-bleed | 218700 | 1968300 | 1968300 | close to 1968300 |

When the serum titer was sufficiently high, a final boost with 400 ug DNA via hydrodynamic tail vein and 4×10⁶ MCF-7 cells via foot pad and subcutaneous was performed before fusion. The lymph nodes and spleen were used for cell fusion.

### 2.2 Hybridoma Generation

Lymph nodes and spleens from immunized mice were homogenized and filtered to remove blood clots and cell debris. Sp2/0 myeloma cells in logarithmic growth were collected and centrifuged. B cells were fused with Sp2/0 myeloma cells at 1:1 ratio in electric fusion solution following general electro-fusion procedures. The fused cells were suspended in DMEM medium supplemented with 20% FBS and IX HAT, and then transferred into 96-well plates (CORNING). The fused cells were cultured in an incubator set to 37° C., 5% CO₂ for 10˜14 days.

### 2.3 Antibody Screening

First screening: Cell-based ELISA assay was used as first screening to test the binding of hybridoma supernatants to human 4IgB7H3. Briefly, human 4IgB7H3 transfected CHOK1 cell line were seeded in 96-well plates (CORNING) at the density of 5×10³ cells/well and were then kept in an incubator set to 37° C., 5% CO₂ for 2 days. The plates were then washed and incubated with hybridoma supernatant for 1 hour at ambient temperature. Plates were then washed and subsequently incubated with secondary antibody goat anti-mouse IgG-Fc-HRP (Bethyl, A90-231P) for 1 hour. After washing, TMB substrate was added and the interaction was stopped by 2 M HCl. The absorbance at 450 nm was read using a microplate reader (Molecular Device).

Second screening: In order to confirm the binding of hybridoma supernatants to human and cynomolgus monkey antigen, FACS was performed. Parental CHOK1 cell line and 4IgB7H13-transfected CHOK1 cell line dyed with CellTrace dye (Violet and FarRed, respectively), while B7H3-transfected FlipinCHO cell pool made with no dye. After incubation for 15 minutes at ambient temperature and wash, equivalent mix three kinds of cells to 1×10⁶ cells/mL. The three cells mixture were transferred into 96-well U-bottom plates (BD) at the density of 1×10⁵ cells/well. The hybridoma supernatants were then transferred to the plates and were incubated for 1 hour at 4° C. After washing, the secondary antibody goat anti-mouse IgG Fc PE (Jackson, 115-115-164) was added and incubated with cells at 4° C. in the dark for 0.5 hour. The cells were then washed and re-suspended in 1×PBS/1% BSA before being analyzed by flow cytometry.

In order to test the binding of hybridoma supernatants to human 2IgB7H3, traditional ELISA assay was used. Plates were pre-coated with 100 μL/well of human 2IgB7H3 (0.2 ug/mL) for 1 hour at ambient temperature. After blocking with 200 μL/well of 1×PBS/2% BSA for 1 hour, the plates were washed with 1×PBST for 3 times. The hybridoma supernatants were added into the plates in a volume of 100 μL/well and incubated for 2 hours at ambient temperature. After washing the plates 3 times with 1×PBST, 100 μL/well of goat anti-mouse IgG-Fc HRP (Bethyl, A90-231P) was added into the plates and incubated for 1 hour at ambient temperature. After washing 6 times, the color was developed by dispensing 100 μL/well of TMB substrate for 5-10 minutes, and then the reaction was stopped by adding 100 μL/well of 2 M HCl. The absorbance was read at 450 nm using a microplate reader.

The selected positive lines screened by FACS were further confirmed by SPR (surface plasmon resonance). SPR allows real-time, label-free detection of bio-molecular interactions. SPR occurs when polarized light strikes an electrically conducting surface at the interface between two media. This generates electron charge density waves called plasmons, reducing the intensity of reflected light at a specific angle known as the resonance angle, in proportion to the mass on a sensor surface.

Biacore 8K was used to rank the binding of supernatants to human 4IgB7H3. The activator was prepared by mixing 400 mM EDC and 100 mM NHS (GE) immediately prior to injection. The CM5 sensor chip was activated for 420 seconds with the activator. Goat anti-mouse Fc IgG (30 ug/mL in 10 mM NaAc, pH 4.5) was then injected to the channel for 420 seconds at a flow rate of 10 μL/min. The chip was deactivated by 1 M ethanolamine-HCl. The supernatants were injected to the channel at a flow rate of 10 μL/min for 30 seconds. 200 nM analyte of human 4IgB7H3 was injected to the channel at a flow rate of 30 μL/min for an association phase of 120 seconds, followed by 300 seconds dissociation. Glycine (10 mM, pH 1.5) as regeneration buffer was injected following dissociation phase. The sensorgrams are subtracted with reference channel and the buffer channel. The data is then fitted with 1:1 Langmuir binding model.

pHrodo iFL dyes can be conjugated to biomolecules such as antibodies, and become highly fluorescent only when found in acidic environments, such as those of cell lysosomes and endosomes. This enables detection of the conjugated biomolecule in the live cell assay. AffiniPure F(ab′)² Fragment goat anti-human IgG Fcγ and anti-mouse IgG Fcγ were labeled with pHrodo iFL Red STP ester amine reactive dye and purified by zeba spin desalting column according to the manufacturer's instructions. MCF-7 cells expressing human full-length B7H3 (2×10+ cells/well) were seeded into 96-well clear bottom black plates pre-coated with 8 μg/mL Poly-D-Lysine. The cells were grown in an incubator set to 37° C., 5% CO₂ overnight. WBP301088-1 positive lines supernatants were added into the corresponding wells in a volume of 100 μL on the next day. The cells were incubated in an incubator set to 37° C., 5% CO₂ for 0.5 hour. Human and mouse IgG1 isotype antibody were used as the negative control. 50 nM pHrodo iFL Red/F(ab′)² goat anti-mouse or anti-human IgG Fcγ conjugates in a volume of 100 μL/well (diluted with complete culture medium) was added. The plates were then incubated in an incubator set to 37° C., 5% CO₂ for 5 hours in the dark. After incubation, the supernatant was discarded. The plates were washed with complete culture medium. The mixed dye solution (1 μg/mL Hoechst 33342 and 0.5 μg/mL Calcein AM diluted in DPBS) was added into the plates in a volume of 100 μL/well and kept in ambient temperature for 15 minutes. After incubation, the dye solution was discarded and the plates were washed once with 1×PBS/1% BSA. The fluorescence intensity of MCF-7 cells was read and analyzed by Perkin Elmer Operetta CLS High-Content Analysis System.

Totally 8460 wells were tested in primary screen and 279 hybridomas were selected and expanded in 24 well plates. Supernatants from the 24-well plates were used to confirm strong binding against human 4IgB7H3 and cynomolgus monkey B7H3, weak binding to human 2IgB7H3 and strong internalization activity on MCF-7 in the secondary screen. Based on the secondary screen result, WBP301088-1.145 hybridoma was selected for subcloning.

### 2.4 Antibody Subcloning

Selected positive hybridoma cells were used for subcloning. The cells in logarithmic growth were counted and were added to 1.5 mL semi-solid-HAT media. The cells were mixed gently in vortex oscillators for 5-10 seconds and then seeded in 6-well plates (CORNING). The plates were kept in an incubator set to 37° C., 5% CO₂ for 7˜8 days. Each visible single colony was picked into 96-well plates (CORNING) with DMEM medium supplemented with 10% FBS. After 2˜3 days, the cell supernatants were collected and screened by cell-based ELISA (human 4IgB7H3), traditional ELISA (human 2IgB7H3) using protocols described as antibody screening above. After subcloning confirmative screening on human 4IgB7H3 and cynomolgus monkey B7H3 by FACS, the culture supernatants of selected single positive clones were collected, and the antibodies were purified for further characterization. WBP301088-1.145.16 was obtained after subclone screen and confirmative screen.

### 2.5 Antibody Isotyping

Isotyping ELISA was performed in 96-well high binding plates (Nunc). Each well was coated with 2 μg/mL capture antibodies goat anti-mouse IgG1 (Bethyl, A90-205A), goat anti-mouse IgG2a (Bethyl, A90-207A), goat anti-mouse IgG2b (Bethyl, A90-209A), goat anti-mouse IgG3 (Bethyl, A90-211A), respectively at 4° C. overnight and blocked with 1×PBS/2% BSA. Hybridoma supernatants were incubated in the coated wells, and specific binding of antibodies to the immobilized capture antibodies on the plates was measured using peroxidase-conjugated goat anti-mouse kappa/lambda light chain antibody. The HRP signal was detected by adding TMB substrate and the reaction was stopped after 12 minutes using 2 M HCl. All incubation steps were performed at ambient temperature, and the plates were washed with PBS between steps. The absorbance at 450 nm was read using a microplate reader (Molecular Device).

The isotyping result of WBP301088-1.145.16 was shown in the table below (Table 4).

TABLE 4
The isotyping result of WBP301088-1.145.16

| Clone No. | Isotype |
|---|---|
| WBP301088-1.145.16 | IgG2b, Kappa |

### 2.6 Antibody Production

T75 flasks (CORNING) are commonly used for scale-up of hybridoma cell culture when small amounts of purified antibody will be needed. Monoclonal hybridoma cells were seeded to the flask at 5×10⁶ to 1×10⁷ cells/T75-flask. The cells were allowed to continually grow about 7-10 days until cell viability reached about 30-40%. Culture supernatant was harvested and cell debris was removed by centrifugation. The supernatant was sterile-filtered and saved for antibody purification.

### 2.7 Hybridoma Sequencing

Total RNA of the hybridoma cell sample was first extracted, following the instruction of the TaKaRa MiniBEST Universal RNA Extraction Kit. The SMART RACE cDNA Amplication Kit from Clonetech was then used to convert RNA to cDNA. The VH and VL domain DNA sequences were then amplified from cDNA with 30 cycles of PCR, each with denaturation at 94° C. for 30 seconds, anneal at 60° C. for 30 seconds, then elongation at 72° C. for 30 seconds. The PCR product was then sub-cloned to TA-cloning vector, then was sent for Biosune Biotech (Shanghai, China) for sequencing.

Once sequencing data confirms monoclonality of the hybridoma cell sample, the DNA sequence of the VH and VL domain was amplified by PCR. The primer was synthesized by Sangon Biotech (Shanghai, China). The DNA segments were then sub-cloned into pcDNA3.4 expression vectors with constant region of human IgG1 with LALA mutations and then were sequenced by Tsingke Biotechnology (Beijing, China).

### 2.8 IgG Conversion

Once sequencing data confirms monoclonality of the hybridoma cell sample, the amino acid sequences of the VH and VL domains were codon optimized for mammalian expression and then synthesized by Sangon Biotech (Shanghai, China). The DNA segments were then sub-cloned into pcDNA3.4 expression vectors with constant region of human IgG1 with L234A/L235A (LALA) mutations.

In this manner, W301088-1.145.16 was converted to human IgG1 with LALA mutation format to obtain a chimeric antibody W301088-1.145.16-xIgG1KV320. After Protein A purification and buffer exchange, it was characterized by SDS-PAGE and SEC-HPLC. The protein migrates with the apparent molecular mass of 50 kDa and 25 kDa on SDS-PAGE under reducing condition, corresponding to the IgG heavy chain and light chain (Data not shown). The purity was higher than 99% (SEC-HPLC).

TABLE 5
Summary of chimeric antibody production.

| | Concen- | | Purity by | |
|---|---|---|---|---|
| | tration | | SEC-HPLC | Theoretical |
| Protein Name | (mg/mL) | Buffer | (%) | M.W. |
| W301088-1.145.16- | 2.50 | PBS | 99.64% | 147 kDa |
| xIgG1KV320 | | | | |

### 2.9 Humanization

A local copy of the open sourced software ANARCI (Dunbar J, Deane CM. ANARCI: antigen receptor numbering and receptor classification. Bioinformatics, 2015; 32 (5): 298-300) was run to assign the Kabat numberings on W301088-1.145.16. The CDRs were identified according to the definitions at Dr. Martin's website (Martin, 2018, supra), which were shown in Table 6.

TABLE 6
Definition of CDR ranges according to Dr.
Martin's antibody informatics website

| CDR | Kabat | Residues before | Residues after | Length |
|---|---|---|---|---|
| L1 | L24-L34 | Always a C | Always a W | 10 to 17 residues |
| L2 | L50-L56 | 16 residues | / | 7 residues |
| | | after L1 | | |
| L3 | L89-L97 | Always a C | Always | 7 to 11 residues |
| | | | F-G-X-G | |
| H1 | H31-H35b | Always | Always a W | 10 to 12 residues |
| | | C-X-X-X | | |
| H2 | H50-H65 | Typically | K/R-L/I/V/F/ | 16 to 19 residues |
| | | L-E-W-G | T/A-T/S/I/A | |
| H3 | H95-H102 | Typically | Always | 3 to 25 residues |
| | | C-A-R | W-G-X-G | |

TABLE 7
VH and VL amino acid sequences of W301088-1.145.16 with underlined CDR
sequences

| Clone | | |
|---|---|---|
| ID | | Amino acid sequence |
| W301088- | VH | DVQLQESGPGLVKPSQSLSLTCTVTDYSITGDYAWNWIRQFPGNKLEWMGYISYS |
| 1.145.16 | | GSTSYNPSLQSRISITRDTSKNQFFLQLNSVTSEDTATYFCARSLGRRWYFVVWGA |
| | | GTTVTVSA |
| | VL | DIVMTQSPSSLAMSVGQKVTMSCKSSQSLLNSSNQKNYLAWYQQKPGQSPKLLIY |
| | | FASTRESGVPDRFIGSGSGTDFTLTISSVQAEDLTDYFCQQHYSAPWTFGGGTKLEI |
| | | K |

TABLE 8
Identified Heavy chain and Light chain CDR sequences of W301088-1.145.16

| Antibody | HCDR1 | HCDR2 | HCDR3 | LCDR1 | LCDR2 | LCDR3 |
|---|---|---|---|---|---|---|
| W301088- | SEQ ID | SEQ ID | SEQ ID | SEQ ID | SEQ ID | SEQ ID |
| 1.145.16 | No: 1 | No: 2 | No: 3 | No: 7 | No: 5 | No: 6 |
| | DYSITG | YISYSG | SLGRRW | KSSQSLL | FASTRES | QQHYSA |
| | DYAW | STSYNP | YFVV | NSSNQK | | PWT |
| | N | SLQS | | NYLA | | |

The VH and VL domain sequences of the murine antibody W301088-1.145.16 were aligned to the human germline sequence repertoires of the VH and VL domains at IMGT, respectively. The human germline sequence of the VH/VL domain with the least number of amino acid differences in framework with respect to the VH/VL domain sequence of W301088-1.145.16 was selected as the humanization template of the VH/VL domain. IGHV4-31*02 combined with IGHJ6*01 and IGKV4-1*01 combined with IGKJ4*01 were the human germline sequences most homologous to the VH and VL domain sequences of W301088-1.145.16, respectively. CDRs of W301088-1.145.16 were grafted into the frameworks of these two human germline templates to constitute the germline sequences.

Several “back mutation” positions in the framework were empirically selected to convert the amino acids in the germline sequence to their counterpart amino acids in the original murine sequence. A set of humanization variants were empirically designed to explore different combinations of these selected back mutation sites. The germline sequences differed from the original murine W301088-1.145.16 sequences at 16 positions in the VL domain and at 20 positions in the VH domain. Among the total 36 different sites, 14 sites were considered for back mutations: VI: Q001D, S025T, H040F, K043N, G044K, 1048M, V067I, V071R, A085E, V089T, Y091F, VL: 1021M, P043S, Y087F. A set of humanization variants were designed empirically to test different combinations of these back mutations.

### 2.11 PTM Removal

The VI and VL domain sequences of the humanization variants were scanned for several types of critical post-translational modification (PTM) sites: asparagine deamination (N-G and N-S) in CDRs, aspartate isomerization (D-G) in CDRs, unpaired C in the entire length, and N-linked glycosylation sites (N-X-S/T in which X can be any amino acid except for P) in the entire length. Point mutations were designed empirically to remove these critical PTM sites in the humanization variants to avoid the potential risk of PTM modification. Finally, we selected the PTM-removal antibodies that do not affect the expression, binding and thermostability compared to the parental antibody.

As the VL domain sequence of the murine antibody W301088-1.145.16 contains N-S-S N-linked glycosylation site, three point mutations of VL: N027dQ, VL: S027 cP and VL: S027fA (according to Kabat numbering) were designed to remove critical PTM site. They were further characterized by k_{off} ranking in conjunction with the humanization variants.

2.12 k_{off} ranking by SPR

The k_{off} ranking of filtered supernatants was performed on a Biacore 8K equipment. CM5 sensor chips were first activated by 400 mM EDC and 100 mM NHS (GE) for 420 seconds at the flow rate of 10 μL/min. 30 μg/mL of anti-human Fc IgG (Jackson) in 10 mM NaAc (pH 4.5) was then injected to channel 1 to 8 at the flow rate of 10 μL/min for 420 seconds. The chips were then deactivated by 1 M ethanolamine-HCl (GE) at the flow rate of 10 μL/min for 420 seconds. Diluted antibody supernatants were injected to Fc2 of the channels at a flow rate of 10 μL/min. Supernatant of the cell culture devoid of plasmid transfection was used as negative control and was injected to Fc1 of the channels at a flow rate of 10 μL/min. 100 nM of human 4IgB7H3 and running buffer were injected orderly to Fc1-Fc2 of the channels at a flow rate of 30 μL/min for an association phase of 180 seconds, followed by dissociation phase of 3600 seconds. 10 mM glycine (pH 1.5) was injected to regenerate the chip after each run. The sensorgrams were analyzed by the software bundled with the Biacore 8K equipment. The sensorgrams of the samples were subtracted with reference channel and buffer channel. The data was then fitted with 1:1 Langmuir binding model.

2.13 Combination of PTM Removal Mutations with Humanization Variants

The PTM removal point mutation showing the best k_{off} rate was combined into the humanization variants with the most promising k_{off} rate by point mutagenesis to constitute the final constructs for affinity validation.

From the k_{off} rates, the PTM removal mutation of VL: N027dQ was combined with four selected humanization mutation amino acids S025T, V071R, Y091F, V089T to constitute the final lead. The final lead was designated as W301088-1.145.16-z3-p1-uIgG1KV320.

TABLE 9
Heavy chain and Light chain CDR sequences of W301088-1.145.16-z3-p1-
uIgG1KV320

| Antibody | HCDR1 | HCDR2 | HCDR3 | LCDR1 | LCDR2 | LCDR3 |
|---|---|---|---|---|---|---|
| W301088- | SEQ ID | SEQ ID | SEQ ID | SEQ ID | SEQ ID | SEQ ID |
| 1.145.16- | No: 1 | No: 2 | No: 3 | No: 4 | No: 5 | No: 6 |
| z3-p1- | DYSIT | YISYSG | SLGRR | KSSQSL | FASTRE | QQHYSA |
| uIgG1KV320 | GDYA | STSYN | WYFVV | LQSSNQ | S | PWT |
| | WN | PSLQS | | KNYLA | | |

### 2.14 Production Scale Transient Transfection and Purification

Each plasmid at the final concentration of 1 μg/mL was transiently transfected to 20 mL Expi293F cells at a density of 3.0×10⁶ cells/mL and cell viability of more than 95% using the ExpiFectamine™ transient transfection kit. The cell culture was grown in a humidified platform shaker with the rotation rate at 150 rpm. The temperature was maintained at 37° C. while the CO₂ level was maintained at 8%.

After 5 days of cell culture incubation, the supernatant expressing the target antibody was collected and filtered for purification using the GE MabSelect SuRE Protein A column (Cytiva, 175438). Eluted antibody was dialyzed into PBS via D-Tube Dialyzer Maxi (EMD Millipore, 71508, MWCO 3.5 kDa). Antibody concentration was determined by absorbance at 280 nm on a NanoDrop device. 2 μg of the purified antibody sample was run on the SDS-PAGE gel (Invitrogen NuPAGE™ 4-12% Bis-Tris Protein Gel) with and without reducing reagent. The purity of the purified antibody sample was quantified by HPLC-SEC with the TSKgel G3000SWXL size exclusion column (Tosoh, 008541). Purified antibody is stored at −80° C.

### Example 3

### In Vitro Characterization of WBP301088 Antibodies

### 3.1 SDS-PAGE and Purity Detection by SEC-HPLC

The samples were mixed with the loading buffer, and heated at 75° C. for 10 minutes using dry bath. After sample loading, SDS-PAGE was run at a constant voltage (200V) for 35 minutes. The gel was stained and destained with eStain™ L1. SDS-PAGE result was obtained using BIO-RAD Imaging System.

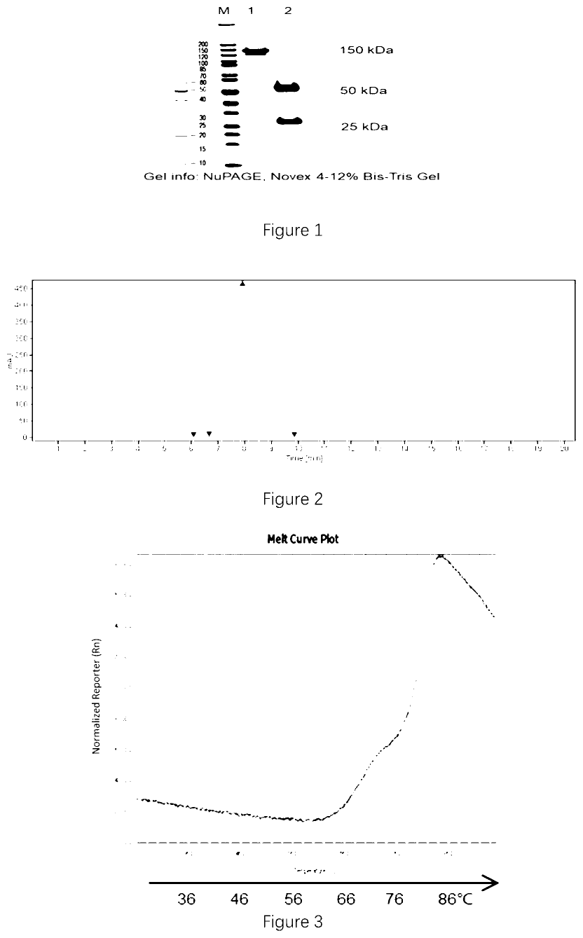

The results are summarized in Table 10 below. The protein migrates with the apparent molecular mass of 50 kDa and 25 kDa on SDS-PAGE under reducing condition, corresponding to the IgG heavy chain and light chain while migrates with expected ˜150 kDa band under non-reducing condition (FIG. 1).

### 3.2 Purity Detection by SEC-HPLC

Purity of each antibody was detected by Agilent 1260 Infinity II system (Agilent Technologics™) with TSKgel G3000SWXL column (Tosoh Bioscience, 0008541). Sample of appropriate volume (5-100 μL) was injected into the column, and separated with a flow rate of 1 mL/min for 20 minutes. The running buffer is 50 mM sodium phosphate, 150 mM NaCl, pH7.0. The peak retention was detected with UV light of the wavelength at 280 nm. The purity of each antibody was analyzed with SEC-HPLC analysis method to integrate all peak areas from 4.5 minutes to 10.5 minutes. The operation and analysis software is the OpenLab CDS Workstation (v2.3.0.443 or v2.2.0.484).

The results are summarized in Table 10 below. The purity of final lead was higher than 99% (SEC-HPLC, FIG. 2).

### 3.3 Thermal Stability (Tₘ) by DSF

Tₘ (melting temperature) of each antibody was investigated using QuantStudio® 7 Flex Real-Time PCR system (Applied Biosystems). 19 μL of antibody solution was mixed with 1 μL of SYPRO Orange solution (Invitrogen) and transferred to the 96 well plate (Applied Biosystems). The plate was sealed with the Optical Adhesive Film (Applied Biosystems) and centrifuged at 3,000 rpm for 5 minutes to remove any air bubbles. The plate was heated from 26° C. to 95° C. at a rate of 0.9° C./min, and the resulting fluorescence data was collected. The negative derivatives of the fluorescence changes with respect to different temperatures were calculated, and the maximal value was defined as melting temperature Tₘ. If a protein has multiple unfolding transitions, the first Tₘ was reported, named as Tₘ1. Data collection and Tₘ calculation were conducted automatically by the QuantStudio® Real Time PCR software (v1.3).

Tₘ value of the protein W301088-1.145.16-7.3-p1-uIgG1KV320 was defined as the maximal value of the negative derivatives of the fluorescence changes with respect to different temperatures, as listed in Table 10 below. Data profile was shown in FIG. 3, in which the dash line indicates the position of the Tₘ1 value in the fluorescence curve. Tₘ1 of W301088-1.145.16-73-p1-uIgG1KV320 is 70.1° C., which indicated it has a good thermal stability.

### 3.4 Hydrophobicity Detection by HIC-HPLC

Hydrophobicity property of antibody was detected by HPLC 1260 Infinity II system (Agilent Technologies™) with TSKgel butyl-NPR column (Tosoh, 0042168). Each sample was diluted to 0.5 mg/mL with PBS buffer and 20 μL diluted sample was injected into the column, and separated with a flow rate of 0.5 mL/min for 61 minutes. The running buffer is 25 mM sodium phosphate, pH 7.0 (Buffer A) and 25 mM sodium phosphate, 1.5 M (NH₄)₂SO₄, pH7.0 (Buffer D). The running gradient was 0% to 100% Buffer D from 3- to 53-minute. The peak retention was detected with UV light of the wavelength at 280 nm and 230 nm. The retention time was analyzed with HIC-HPLC analysis method to integrate all peak areas from 20- to 40-minute. The operation and analysis software is the OpenLab CDS Workstation (v2.6.0.691).

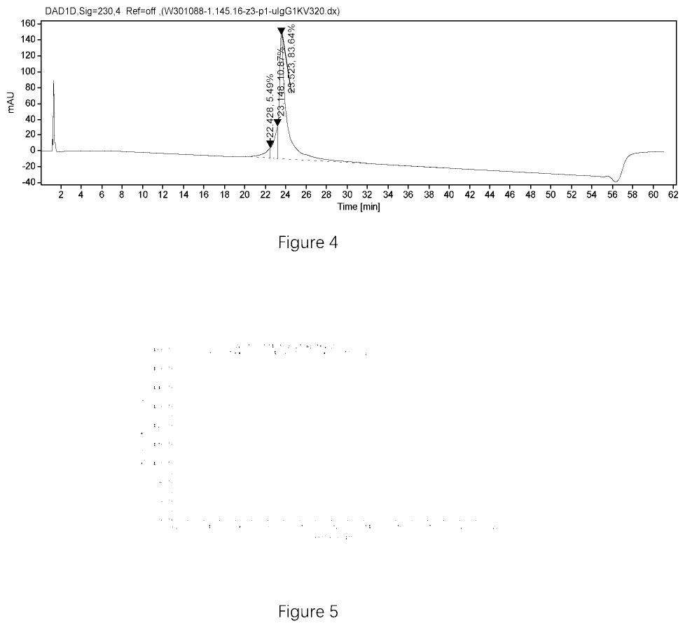

The HIC retention time of the protein W301088-1.145.16-73-p1-uIgG1KV320 is 23.52-minute (Table 10 and FIG. 4), which indicated it's an antibody with good hydrophilicity.

### 3.5 Diffusion Interaction Parameter (k_{D}) by DLS

DLS-kp measurement was investigated using DynaPro Plate Reader III (Wyatt Technology). During the sample preparation process, the appearance of samples was recorded at thawing, filtration and concentration. Samples were concentrated to over 20 mg/mL and then diluted with PBS Buffer to a final concentration at 2.5, 5, 10, 15, and 20 mg/mL. 7.5 AL sample solution was then added to 1536 well microplate (Aurora, ABI1-00110A). The plate was sealed with the ClearSeal Film (Hampton Research, HR4-521), and centrifuged at 3,000 rpm for 5 minutes to let the sample down to the bottom of the well. Each sample was tested in duplicate wells. The plate was put into the corresponding position and data collection was performed by the DYNAMICS operation software (v7.8.1.3). 5 acquisitions were collected for each protein sample while each acquisition time was 5 seconds. For each measurement, the diffusion coefficient was determined and plotted against protein concentration. k_{D} values were calculated automatically by the software (v7.8.1.3), as listed in Table 10. Data profiles were shown in FIG. 5. The k_{D} of the protein W301088-1.145.16-73-p1-uIgG1KV320 in PBS is-5.17 mL/g, which represents it's an antibody with high solubility. During the assay process, appearance of the protein was also recorded, a few particles were observed after thawing and gently shaking. If the buffer was exchanged to 20 mM His, 8% sucrose, 0.02% PS80, pH6.5, no particle was observed.

The above described SDS-PAGE, SEC-HPLC, HIC-HPLC, DSF and DLS results were summarized in Table 10.

TABLE 10
The analysis data summary of W301088-1.145.16-z3-p1-uIgG1KV320

| | Purity by | Yield | Molecular | | HIC Retention time | Tₘ | DLS-k_{D} |
|---|---|---|---|---|---|---|---|
| Antibody Name | SEC-HPLC | (mg/L) | Weight | pI | (min) | (° C.) | (mL/g) |
| W301088- | 99.26% | 416.0 | 147 kDa | 8.54 | 23.52 | 70.1 | −5.17 |
| 1.145.16-z3-p1- | | | | | | | |
| uIgG1KV320 | | | | | | | |

### 3.6 Human B7H3 Binding (FACS)

FACS was used to detect the binding of antibodies to human B7H3. This method can quantitatively analyze and identify specific molecules expressed on the surface of living cells. Unlabeled cells were used as a control to set the threshold before detection, and the percentage change of each group that exceeded the fluorescence intensity threshold was analyzed. MCF-7 cells expressing human full-length B7H3 (1×10⁵ cells/well) were incubated with various concentrations of the antibodies (3.16-fold serially diluted with 1×DPBS/1% BSA from 50 nM to 0.0005 nM) in a volume of 100 L/well for 1 hour in a refrigerator set to 4° C. Anti-human B7H3 reference antibody, Enoblituzumab (MacroGenics) was used as the positive control. Human IgG1 isotype antibody was used as the negative control. After washing the cells with 1×DPBS/1% BSA, Alexa 647 goat anti-human antibody (diluted 1:500 in 1×DPBS/1% BSA) was added. The cells were incubated in a refrigerator set to 4° C. for 0.5 hour in the dark. The mean fluorescence intensity (MFI) of the cells was measured by a flow cytometer and analyzed by FlowJo. The EC₅₀ values were calculated by four-parameter non-linear regression analysis using GraphPad Prism 7 software.

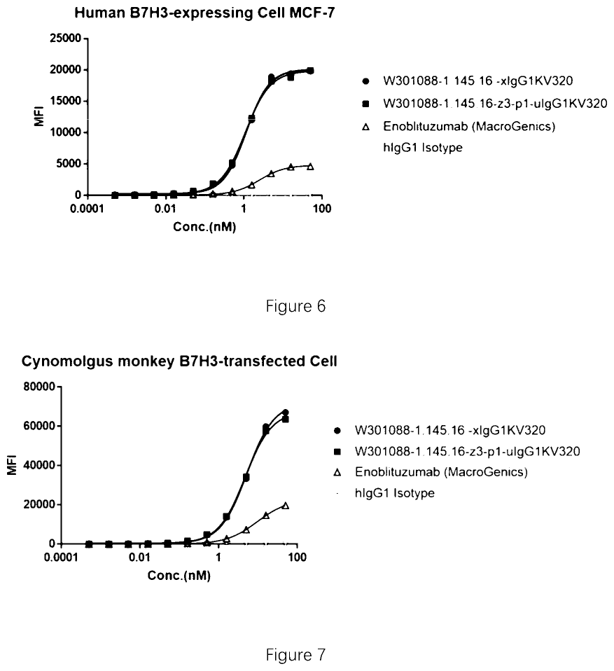

W301088-1.145.16-73-p1-uIgG1KV320 showed good binding to B7H3-expressing MCF-7 cell with an EC₅₀ of 1.08 nM and max MFI of 19900, which was comparable with chimeric antibody and better than reference antibody Enoblituzumab (MacroGenics) (Table 11, FIG. 6).

TABLE 11
FACS binding data on human B7H3 expressing cell MCF-7

MCF-7

| Samples | EC₅₀ (nM) | Max MFI |
|---|---|---|
| W301088-1.145.16-xIgG1KV320 | 1.16 | 19800 |
| W301088-1.145.16-z3-p1-uIgG1KV320 | 1.08 | 19900 |
| Enoblituzumab (MacroGenics) | 2.49 | 4644 |
| hIgG1 Isotype | N.A. | 47.8 |

Note:
N.A. means not available.

### 3.7 Cynomolgus Monkey B7H3 Binding (FACS)

FACS was used to detect the binding of antibodies to cynomolgus monkey B7H3. FlpinCHO cells were transfected with cynomolgus monkey B7H3 (W3XX088-FlpinCHO.cProl.pool) (1×10⁵ cells/well) and incubated with various concentrations of the antibodies (3.16-fold serially diluted with 1×DPBS/1% BSA from 50 nM to 0.0005 nM) in a volume of 100 μL/well for 1 hour in a refrigerator set to 4° C. Anti-human B7H3 reference antibody, enoblituzumab (MacroGenics) was used as the positive control. Human IgG1 isotype antibody was used as the negative control. After washing the cells with 1×DPBS/1% BSA, Alexa 647 goat anti-human antibody (diluted 1:500 in 1×DPBS/1% BSA) was added. The cells were incubated in a refrigerator set to 4° C. for 0.5 hour in the dark. The mean fluorescence intensity (MFI) of the cells was measured by a flow cytometer and analyzed by FlowJo. The EC₅₀ values were calculated by four-parameter non-linear regression analysis using GraphPad Prism 7 software.

W301088-1.145.16-73-p1-uIgG1KV320 showed good binding to cynomolgus monkey transfected cell with an EC₅₀ of 4.68 nM, which was comparable with chimeric antibody, better than enoblituzumab (MacroGenics) (Table 12, FIG. 7).

TABLE 12
FACS binding data on cynomolgus monkey
B7H3 transfected stable cell pool

| | Cynomolgus monkey B7H3- | |
|---|---|---|
| | transfected stable cell pool | |
| Samples | EC₅₀ (nM) | Max MFI |
| W301088-1.145.16-xIgG1KV320 | 5.25 | 67100 |
| W301088-1.145.16-z3-p1-uIgG1KV320 | 4.68 | 63600 |
| Enoblituzumab (MacroGenics) | 9.58 | 19700 |
| hIgG1 Isotype | N.A. | 6.67 |

Note:
N.A. means not available.

### 3.8 Human 4IgB7H3 Binding (SPR)

SPR was used to determine the affinity of antibodies to human 4IgB7H3. The affinity of antibodies to human 4IgB7H3 was tested by Biacore 8K. The activator was prepared by mixing 400 mM EDC and 100 mM NHS (GE) immediately prior to injection. The CM5 sensor chip was activated for 420 seconds with the activator. Goat anti-human Fc IgG (30 ug/mL in 10 mM NaAc, pH 4.5) was then injected to the channel for 420 seconds at a flow rate of 10 μL/min. The chip was deactivated by 1 M ethanolamine hydrochloric acid. Diluted antibodies in running buffer (1×HBS-EP+) were injected to the channel at a flow rate of 10 μL/min for 30 seconds. Seven concentrations (100, 50, 25, 12.5, 6.25, 2.125 and 1.563 nM) of analyte human 4IgB7H3 were injected orderly to the channel at a flow rate of 30 μL/min for an association phase of 180 seconds, followed by 3600 seconds dissociation. Glycine (10 mM, pH 1.5) as regeneration buffer was injected following dissociation phase.

The sensorgrams for reference channel and buffer channel were subtracted from the test sensorgrams. The experimental data was fitted with a 1:1 binding model. The molecular weight of the human 4IgB7H3 used in the calculation was 47.8 kDa.

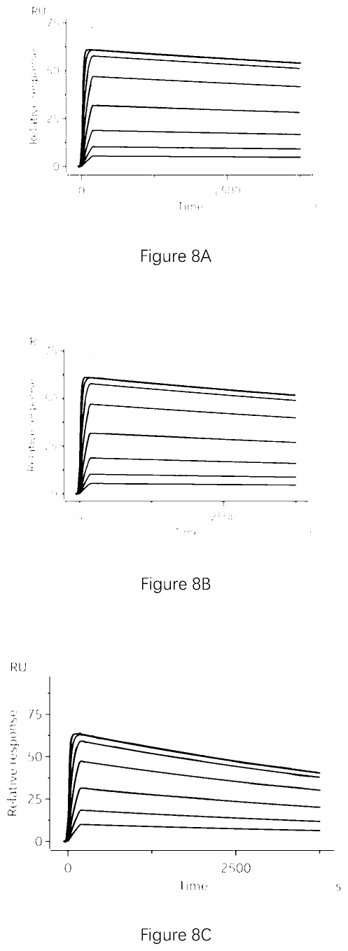

The experimental data was fitted by 1:1 binding model bundled in Biacore 8K evaluation software. As shown in Table 13 and FIGS. 8A-8C, W301088-1.145.16-z3-p1-uIgG1KV320 showed high binding affinity to human 4IgB7H3. The K_{D} value was 7.08E-11 M, which was better than enoblituzumab (MacroGenics).

TABLE 13
Affinity result of antibodies binding to human 4IgB7H3

| Analyte | Ligand | kₐ (1/Ms) | k_{d} (1/s) | K_{D} (M) |
|---|---|---|---|---|
| Human | W301088-1.145.16 - | 6.57E+05 | 3.39E−05 | 5.15E−11 |
| 4IgB7H3 | xIgG1KV320 | | | |
| | W301088- | 6.57E+05 | 4.65E−05 | 7.08E−11 |
| | 1.145.16-z3-p1- | | | |
| | uIgG1KV320 | | | |
| | Enoblituzumab | 1.24E+05 | 1.26E−04 | 1.02E−09 |
| | (MacroGenics) | | | |

### 3.9 Human 2IgB7H3 Binding (SPR)

SPR was used to determine the affinity of antibodies to human 2IgB7H3. The affinity of antibodies to human 2IgB7H3 was tested by Biacore 8K. The activator was prepared by mixing 400 mM EDC and 100 mM NHS (GE) immediately prior to injection. The CM5 sensor chip was activated for 420 seconds with the activator. Goat anti-human Fc IgG (30 μg/mL in 10 mM NaAc, pH 4.5) was then injected to the channel for 420 seconds at a flow rate of 10 L/min. The chip was deactivated by 1M ethanolamine hydrochloric acid. Diluted antibodies in running buffer (1×HBS-EP+) were injected to the channel at a flow rate of 10 μL/min for 30 seconds. Seven concentrations (500, 250, 125, 62.5, 31.25, 15.625 and 7.813 nM) of analyte human 2IgB7H3 were injected orderly to the channel at a flow rate of 30 μL/min for an association phase of 120 seconds, followed by 300 seconds dissociation. Glycine (10 mM, pH 1.5) as regeneration buffer was injected following dissociation phase.

The sensorgrams for reference channel and buffer channel were subtracted from the test sensorgrams. The experimental data was fitted with a 1:1 binding model. The molecular weight of the human 2IgB7H3 used in the calculation was 25.2 kDa.

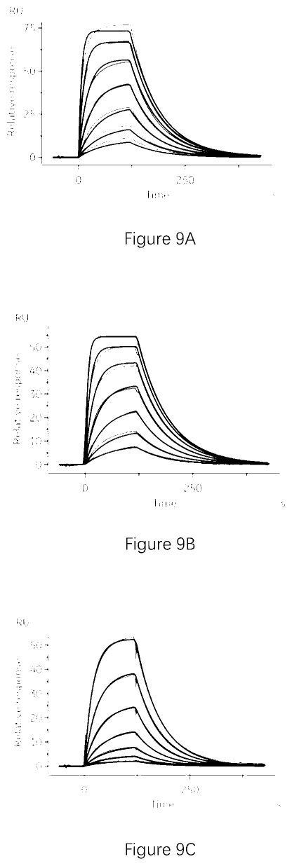

The experimental data was fitted by 1:1 binding model bundled in Biacore 8K evaluation software. As shown in Table 14 and FIGS. 9A-9C, W301088-1.145.16-z3-p1-uIgG1KV320 showed weaker binding affinity to human 21gB7H3 compared to its binding to human 4IgB7H3. The K_{D} value was 4.64E-08 M.

TABLE 14
Affinity result of antibodies binding to human 2IgB7H3

| Analyte | Ligand | kₐ (1/Ms) | k_{d} (1/s) | K_{D} (M) |
|---|---|---|---|---|
| Human | W301088-1.145.16 - | 2.35E+05 | 1.36E−02 | 5.77E−08 |
| 2IgB7H3 | xIgG1KV320 | | | |
| | W301088- | 3.03E+05 | 1.41E−02 | 4.64E−08 |
| | 1.145.16-z3-p1- | | | |
| | uIgG1KV320 | | | |
| | Enoblituzumab | 6.44E+04 | 1.68E−02 | 2.61E−07 |
| | (MacroGenics) | | | |

### 3.10 Cynomolgus Monkey B7H3 Binding (SPR)

SPR was used to determine the affinity of antibodies to cynomolgus monkey B7H3. The affinity of antibodies to cynomolgus monkey B7H3 was tested by Biacore 8K. The activator was prepared by mixing 400 mM EDC and 100 mM NHS (GE) immediately prior to injection. The CM5 sensor chip was activated for 420 seconds with the activator. Goat anti-human Fc IgG (30 ug/mL in 10 mM NaAc, pH 4.5) was then injected to the channel for 420 seconds at a flow rate of 10 μL/min. The chip was deactivated by IM ethanolamine hydrochloric acid. Diluted antibodies in running buffer (1×HBS-EP+) were injected to the channel at a flow rate of 10 μL/min for 30 seconds. Nine concentrations (50, 25, 12.5, 6.25, 3.125, 1.563, 0.781, 0.391 and 0.196 nM) of analyte cynomolgus monkey B7H3 were injected orderly to the channel at a flow rate of 30 μL/min for an association phase of 180 seconds, followed by 3600 seconds dissociation. Glycine (10 mM, pH 1.5) as regeneration buffer was injected following dissociation phase.

The sensorgrams for reference channel and buffer channel were subtracted from the test sensorgrams. The experimental data of 6 analyte concentrations (50, 25, 12.5, 6.25, 3.125 and 1.563 nM) was fitted with a 1:1 binding model. The molecular weight of the cynomolgus monkey B7H3 used in the calculation was 49 kDa.

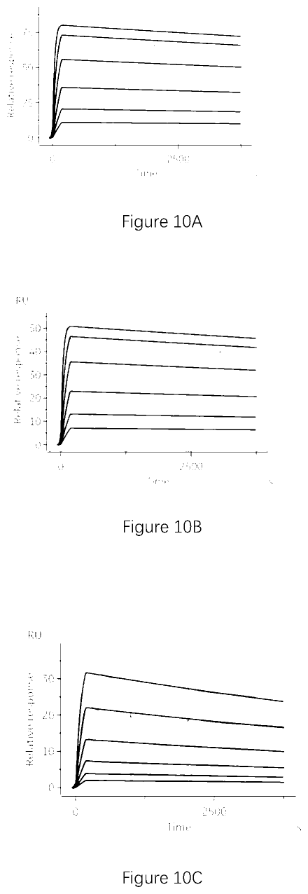

The experimental data was fitted by 1:1 binding model bundled in Biacore 8K evaluation software. As shown in Table 15 and FIGS. 10A-10C, W301088-1.145.16-z3-p1-uIgG1KV320 showed high binding affinity to cynomolgus monkey B7H3, which was comparable to its binding to human 4IgB7H3. The K_{D} value was 5.74E-11 M, which was better than enoblituzumab (MacroGenics).

TABLE 15
Affinity result of antibodies binding to cynomolgus monkey B7H3

| Analyte | Ligand | kₐ (1/Ms) | k_{d} (1/s) | K_{D} (M) |
|---|---|---|---|---|
| cynomolgus | W301088-1.145.16- | 5.21E+05 | 2.94E−05 | 5.64E−11 |
| monkey | xIgG1KV320 | | | |
| B7H3 | W301088- | 5.27E+05 | 3.02E−05 | 5.74E−11 |
| | 1.145.16-z3-p1- | | | |
| | uIgG1KV320 | | | |
| | Enoblituzumab | 1.85E+05 | 8.14E−05 | 4.41E−10 |
| | (MacroGenics) | | | |

### 3.11 Internalization Test (HCS)

Antibody internalization was determined by operetta CLS (PerkinElmer), a high content imaging and analysis system that can collect and analyze images of samples with high speed and sensitivity. MCF-7 cells expressing human full-length B7H3 (1.5×10⁴ cells/well) were seeded into 96-well clear bottom black plates pre-coated with 8 μg/mL Poly-D-Lysine. After incubating in an incubator set to 37° C., 5% CO₂ overnight, various concentrations of the antibodies (3.16-fold serially diluted with 1×DPBS/1% BSA from 50 nM to 0.0005 nM) in a volume of 100 μL/well for 2 hours in a refrigerator set to 4° C. Anti-human B7H3 reference antibody, enoblituzumab (MacroGenics) was used as the positive control. Human IgG1 isotype antibody was used as the negative control. After washing the cells with 1×DPBS/1% BSA, Alexa 647 goat anti-human antibody (diluted 1:500 in 1×DPBS/1% BSA) was added. The cells were incubated in a refrigerator set to 4° C. for 1 hour in the dark. The cells were then washed once and resuspended in 1×DPBS/1% BSA for 2 hours at 37° C. After incubation, supernatants were discarded and 100 μL/well Hoechst 33342 (diluted 1:5000 in 1×DPBS) were added and incubated at ambient temperature for 15 minutes. After washing the cells with 1×DPBS, 100 μL/well acid washing buffer (100 mM glycine, 150 mM NaCl, pH2.5) were added and incubated in a refrigerator set to 4° C. for 5 minutes. The cells were washed once with 1×DPBS and fixed with 60 μL 4% PFA. The mean fluorescence intensity (MFI) of the cells was measured and analyzed by operetta CLS. The EC₅₀ values were calculated by four-parameter non-linear regression analysis using GraphPad Prism 7 software.

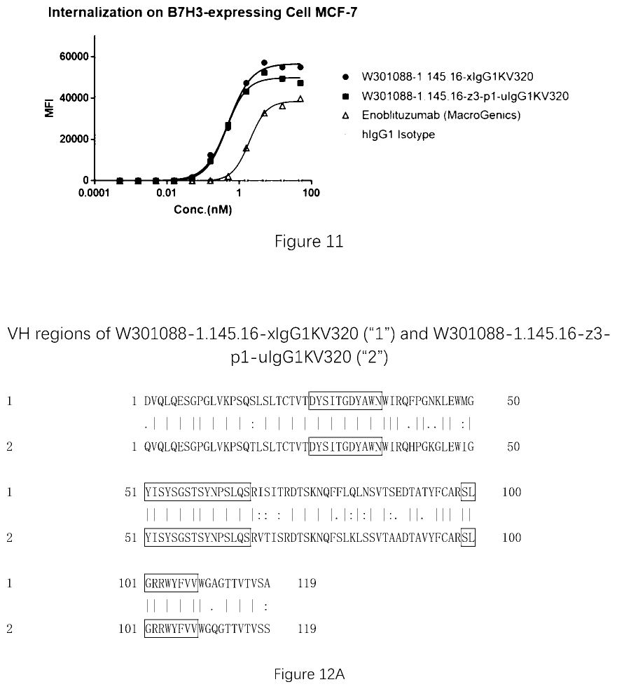

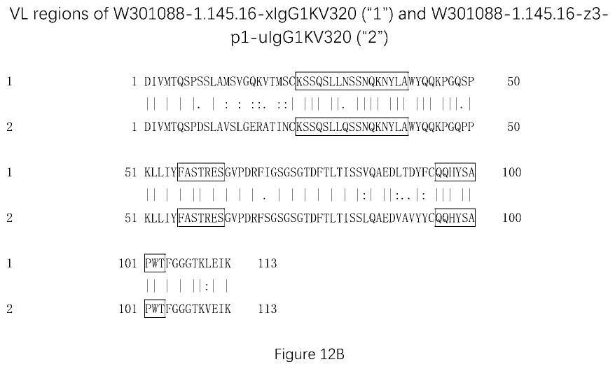

W301088-1.145.16-z3-p1-uIgG1KV320 showed good internalization ability to B7H3-expressing MCF-7 cells with an EC₅₀ of 0.51 nM, which was comparable with chimeric antibody and better than reference antibody Enoblituzumab (MacroGenics) (Table 16, FIG. 11).

TABLE 16
Internalization on human B7H3 expressing cell MCF-7

| | Internalization on MCF-7 | |
|---|---|---|
| Samples | EC₅₀ (nM) | Max MFI |
| W301088-1.145.16-xIgG1KV320 | 0.51 | 57180 |
| W301088-1.145.16-z3-p1-uIgG1KV320 | 0.44 | 52315 |
| Enoblituzumab (MacroGenics) | 1.95 | 39777 |
| hIgG1 Isotype | N.A. | 21 |

Note:
N.A. means not available.

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

What is claimed:

1. An antibody or antigen-binding portion thereof that binds to B7H3, which comprises the following CDRs:
a heavy chain CDR1 (HCDR1) comprising the amino acid sequence of SEQ ID No: 1; a HCDR2 comprising the amino acid sequence of SEQ ID No: 2; a HCDR3 comprising the amino acid sequence of SEQ ID No: 3; and
a light chain CDR1 (LCDR1) comprising the amino acid sequence of SEQ ID No: 4 or 7; a LCDR2 comprising the amino acid sequence of SEQ ID No: 5, and a LCDR3 comprising the amino acid sequence of SEQ ID No: 6.

2. The antibody or antigen-binding portion thereof of claim 1, wherein the antibody or antigen-binding portion thereof comprises a heavy chain variable region (VH) and a light chain variable region (VL), wherein the VH comprises:
(A) an amino acid sequence as set forth in SEQ ID No: 8 or 10;
(B) an amino acid sequence which is at least 85%, at least 90%, or at least 95% identical to SEQ ID No: 8 or 10; or
(C) an amino acid sequence with addition, deletion and or substitution of one or more (for example, 1, 2, 3, 4, 5, 6, 7, 8, 9 or 10) amino acids in the framework region compared with SEQ ID No: 8 or 10;
and/or the VL comprises:
(A) an amino acid sequence as set forth in SEQ ID No: 9 or 11;
(B) an amino acid sequence which is at least 85%, at least 90%, or at least 95% identical to SEQ ID No: 9 or 11; or
(C) an amino acid sequence with addition, deletion and or substitution of one or more (for example, 1, 2, 3, 4, 5, 6, 7, 8, 9 or 10) amino acids in the framework region compared with SEQ ID No: 9 or 11.

3. The antibody or antigen-binding portion thereof of claim 2, wherein the VH and/or VL comprises one or more amino acid substitutions in the framework region.

4. The antibody or antigen-binding portion thereof of claim 3, wherein at least one of the amino acid substitutions is a back mutation wherein an amino acid from human germline sequences is substituted by a different amino acid at corresponding positions in a parental antibody.

5. The antibody or antigen-binding portion thereof of claim 3, wherein at least one of the amino acid substitutions removes a potential post-translational modification site(s).

6. The antibody or antigen-binding portion thereof of any of the preceding claims, wherein the B7H3 is human B7H3 protein, mouse B7H3 protein, cynomolgous monkey B7H3 protein or the extracellular domain thereof.

7. The antibody or antigen-binding portion thereof of claim 6, wherein the B7H3 is human 4IgB7H3 protein or the extracellular domain thereof.

8. The antibody or antigen-binding portion thereof of any of the preceding claims, wherein the antibody or antigen-binding portion thereof is a full antibody, scFv, Fab, F(ab′)2, or Fv fragment.

9. The antibody or antigen-binding portion thereof of any of the preceding claims, wherein the antibody comprises an immunoglobulin constant region, such as a human IgG constant region.

10. The antibody or antigen-binding portion thereof of claim 9, wherein the immunoglobulin constant region is a human IgG1 constant region, optionally comprising one or more modifications such as L234A/L235A (LALA) mutations.

11. The antibody or antigen-binding portion thereof of any of the preceding claims, wherein the antibody or antigen-binding portion thereof is a chimeric antibody or a humanized antibody.

12. The antibody or antigen-binding portion thereof of any of the preceding claims, comprising:
(a) the amino acid sequence of SEQ ID No: 12 for the heavy chain and the amino acid sequence of SEQ ID No: 13 for the light chain; and or
(b) the amino acid sequence of SEQ ID No: 14 for the heavy chain and the amino acid sequence of SEQ ID No: 15 for the light chain.

13. An isolated nucleic acid molecule, comprising a nucleic acid sequence encoding the VH and/or VL region of the antibody or antigen-binding portion thereof of any of claims 1-12.

14. The isolated nucleic acid molecule of claim 13, comprising a nucleic acid sequence as set forth in SEQ ID No: 16 and/or 17.

15. A vector comprising the nucleic acid molecule of claim 13 or 14.

16. A host cell comprising the nucleic acid molecule of claim 13 or claim 14 or the vector of claim 15.

17. A pharmaceutical composition comprising the antibody or antigen-binding portion thereof as defined in any of claims 1-12 and a pharmaceutically acceptable carrier.

18. A method for producing an antibody or antigen-binding portion thereof, comprising the steps of:
culturing the host cell of claim 16 under suitable conditions for expression of the antibody or antigen-binding portion thereof; and
isolating the antibody or antigen-binding portion thereof from the host cell.

19. A method for modulating a B7H3-related immune response in a subject, comprising administering to the subject the antibody or antigen-binding portion thereof as defined in any of claims 1-12 or the pharmaceutical composition of claim 17 to the subject.

20. A method for preventing or treating a B7H3-related disorder in a subject, comprising administering an effective amount of the antibody or antigen-binding portion thereof as defined in any of claims 1-12 or the pharmaceutical composition of claim 15 to the subject.

21. The method of claim 20, wherein the B7H3-related disorder is selected from cancers, autoimmune diseases and infectious diseases.

22. The method of claim 21, wherein the cancer is selected from breast cancer, neurological tumor, melanoma, lung cancer, head and neck cancer, colorectal cancer, pancreatic cancer, stomach cancer, kidney cancer, bladder cancer, prostate cancer, ovarian cancer, cervical cancer, glioblastoma, esophageal cancer, bladder cancer, renal cell carcinoma, endometrial cancer, skin cancer, testis cancer, thyroid cancer, urothelial cancer, lymphoma such as non-Hodgkin's lymphoma and diffuse large B-cell lymphoma, leukemia such as chronic lymphocytic leukemia, and multiple myeloma.

23. The method of any of claims 21-22, wherein the antibody or antigen-binding portion thereof is administered in combination with cell immunotherapy, chemotherapy, radiation therapy, targeted therapy and or other agents for use in cancer immunotherapy.

24. The antibody or antigen-binding portion thereof as defined in any of claims 1-12 for use in treating or preventing a B7H3-related disorder in a subject.

25. Use of the antibody or antigen-binding portion thereof of any of claims 1-12 in the manufacture of a medicament for diagnosing, treating or preventing a B7H3-related disorder in a subject.

26. A kit comprising the antibody or antigen-binding portion thereof of any of claims 1-12.


SEQUENCE LISTING (17 sequences)

SEQ ID NO: 1 - protein, 11 aa, synthetic construct
DYSITGDYAWN

SEQ ID NO: 2 - protein, 16 aa, synthetic construct
YISYSGSTSYNPSLQS

SEQ ID NO: 3 - protein, 10 aa, synthetic construct
SLGRRWYFVV

SEQ ID NO: 4 - protein, 17 aa, synthetic construct
KSSQSLLQSSNQKNYLA

SEQ ID NO: 5 - protein, 7 aa, synthetic construct
FASTRES

SEQ ID NO: 6 - protein, 9 aa, synthetic construct
QQHYSAPWT

SEQ ID NO: 7 - protein, 17 aa, synthetic construct
KSSQSLLNSSNQKNYLA

SEQ ID NO: 8 - protein, 119 aa, synthetic construct
DVQLQESGPGLVKPSQSLSLTCTVTDYSITGDYAWNWIRQFPGNKLEWMGYISYSGSTSYNPSLQSRISITRDTSKNQFFLQLNSVTSEDTATYFCARSLGRRWYFVVWGAGTTVTVSA

SEQ ID NO: 9 - protein, 113 aa, synthetic construct
DIVMTQSPSSLAMSVGQKVTMSCKSSQSLLNSSNQKNYLAWYQQKPGQSPKLLIYFASTRESGVPDRFIGSGSGTDFTLTISSVQAEDLTDYFCQQHYSAPWTFGGGTKLEIK

SEQ ID NO: 10 - protein, 119 aa, synthetic construct
QVQLQESGPGLVKPSQTLSLTCTVTDYSITGDYAWNWIRQHPGKGLEWIGYISYSGSTSYNPSLQSRVTISRDTSKNQFSLKLSSVTAADTAVYFCARSLGRRWYFVVWGQGTTVTVSS

SEQ ID NO: 11 - protein, 113 aa, synthetic construct
DIVMTQSPDSLAVSLGERATINCKSSQSLLQSSNQKNYLAWYQQKPGQPPKLLIYFASTRESGVPDRFSGSGSGTDFTLTISSLQAEDVAVYYCQQHYSAPWTFGGGTKVEIK

SEQ ID NO: 12 - protein, 449 aa, synthetic construct
QVQLQESGPGLVKPSQTLSLTCTVTDYSITGDYAWNWIRQHPGKGLEWIGYISYSGSTSYNPSLQSRVTISRDTSKNQFSLKLSSVTAADTAVYFCARSLGRRWYFVVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 220 aa, synthetic construct
DIVMTQSPDSLAVSLGERATINCKSSQSLLQSSNQKNYLAWYQQKPGQPPKLLIYFASTRESGVPDRFSGSGSGTDFTLTISSLQAEDVAVYYCQQHYSAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 448 aa, synthetic construct
DVQLQESGPGLVKPSQSLSLTCTVTDYSITGDYAWNWIRQFPGNKLEWMGYISYSGSTSYNPSLQSRISITRDTSKNQFFLQLNSVTSEDTATYFCARSLGRRWYFVVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 220 aa, synthetic construct
DIVMTQSPSSLAMSVGQKVTMSCKSSQSLLNSSNQKNYLAWYQQKPGQSPKLLIYFASTRESGVPDRFIGSGSGTDFTLTISSVQAEDLTDYFCQQHYSAPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 357 bp, synthetic construct
  source 1..357: mol_type = other DNA
caggtgcagctccaggagtctggccctggcctggtgaagccatctcagacactgagcctgacatgtaccgtgaccgattatagcatcaccggcgactacgcttggaattggatcagacagcaccctggaaagggcctggagtggattggatacatcagctattctggcagcacatcttataatcctagcctgcagtctagggtgacaatttcaagagataccagcaaaaatcagtttagcctgaaactgagctctgtgaccgccgccgatacagccgtgtatttttgtgccagaagcctgggaagaagatggtattttgtggtgtggggacagggcacaacagtgaccgtgtctagc

SEQ ID NO: 17 - DNA, 339 bp, synthetic construct
  source 1..339: mol_type = other DNA
gatattgtgatgacccagtctcctgatagcctggccgtgagcctgggcgagagagccacaatcaattgtaagtctagccagtctctgctgcagagctctaatcagaaaaattacctggcctggtatcagcagaagcctggccagcctcctaagctgctgatctactttgccagcacaagggaatctggagtgcctgataggtttagcggcagcggctctggcacagattttacactgacaatctctagcctgcaggccgaggatgtggccgtgtattattgtcagcagcactattctgccccttggacatttggcggcggcaccaaagtggagattaag